(19) United States
(12) Patent Application Publication
(10) Pub. No.: US 2025/0250308 A1
(43) Pub. Date: Aug. 7, 2025

(54) METHODS AND COMPOSITIONS WITH ANTIFUNGAL ACTIVITY

(71) Applicant: THE BOARD OF REGENTS OF THE UNIVERSITY OF TEXAS SYSTEM, Austin, TX (US)

(72) Inventors: Danielle A. GARSIN, Houston, TX (US); Shantanu GUHA, Houston, TX (US); Michael C. LORENZ, Houston, TX (US)

(73) Assignee: THE BOARD OF REGENTS OF THE UNIVERSITY OF TEXAS SYSTEM, Austin, TX (US)

(21) Appl. No.: 18/856,196

(22) PCT Filed: Apr. 13, 2023
(86) PCT No.: PCT/US2023/065708

§ 371 Date: Oct. 11, 2024

Related U.S. Application Data
(60) Provisional application No. 63/331,203, filed on Apr. 14, 2022.

(51) Int. Cl. C07K 14/315; A23B 2/729; A61K 38/00; A61K 47/64; A61P 31/10; C09D 5/16

(52) U.S. Cl. CPC C07K 14/315; A23B 2/7295; A61K 47/64; A61P 31/10; C09D 5/165; A61K 38/00; C07K 2319/55

(57) ABSTRACT

Described are methods and compositions that provide for the production and use of generating synthetic or recombinant peptides having antifungal properties. Wherein a peptide or polypeptide consists or consists essentially of an anti-fungal portion of the alpha-7 domain of EntV, wherein the peptide or polypeptide comprises an amino acid sequence.

### PRIORITY CLAIM

This application claims the benefit of U.S. Provisional Patent Application No. 63/331,203, filed Apr. 14, 2022, which is incorporated herein by reference in its entirety.

### STATEMENT REGARDING FEDERALLY SPONSORED RESEARCH OR DEVELOPMENT

This invention was made with U.S. Government support under Grant No. DE027608 awarded by the National Institutes of Health. The government has certain rights in the invention.

### SEQUENCE LISTING

This application contains a Sequence Listing XML, which has been submitted electronically and is hereby incorporated by reference in its entirety. Said XML Sequence Listing, created on Apr. 11, 2023, is named UTFHP0383WO.xml and is 26,302 bytes in size.

### TECHNICAL FIELD

Generally, described herein are methods and antifungal compositions that inhibit fungal growth which have both medical and non-medical utility and applications.

### BACKGROUND

The disclosure relates to synthetic antifungal peptide compositions that inhibit fungal growth which have both medical and non-medical utility and applications. Fungal infections can range from superficial and cutaneous to deeply invasive and disseminated. The management of disseminated fungal infections continues to be a major clinical problem. The limited spectrum of available antifungal agents contributes to both the development of acquired resistance and the rise in incidence of previously rare but intrinsically resistant pathogens (Arastehfar et al., 2020; Perlin et al., 2017). Consequently, there is an unacceptably high mortality rate in patients with fungal infections (Brown et al., 2012). Antifungal discovery is complicated by the similar cell biology of fungi and mammals; as a result, there are only three classes of drugs for systemic infections, the newest of which was discovered nearly 40 years ago and approved for clinical use in 2001 (Perfect, 2017). Thus, there is a longstanding and unmet need for new antifungals for use in both human and veterinary medical fields.

In addition to this unmet medical need, fungal growth on indoor and outdoor surfaces is a major environmental concern affecting home, work and recreational environments. Not only can fungus (e.g., mold, mildew) be unsightly on exposed surfaces, it can destroy wood, fiber and other materials if left untreated, causing severe damage to buildings and other structures and equipment. Furthermore, there is also significant loss due to fungal damage of agricultural and horticultural crops, particularly to seeds, seedlings and agricultural commodities, including mature plants such as trees. Provided herein are compositions and methods antifungal compositions and methods that can be used to inhibit fungal growth with both medical and non-medical utilities and applications.

### SUMMARY

Certain embodiments of the present disclosure provide methods and compositions with antifungal activity. In some embodiments, there are provided peptides or polypeptides consisting or consisting essentially of an anti-fungal portion of the a7 domain of EntV. In some aspects, these peptides or polypeptides consist or essentially consist of an amino acid sequence selected from of SEQ ID NO: 3, SEQ ID NO: 4, SEQ ID NO: 5 and SEQ ID NO: 6. In further aspects the antifungal peptides or polypeptides consist or essentially consist of an amino acid sequence selected from of SEQ ID NO: 7, SEQ ID NO: 8, SEQ ID NO: 9, and SEQ ID NO: 10. In still other aspects, antifungal peptides or polypeptides comprise an amino acid sequence selected from of SEQ ID NO: 7, SEQ ID NO: 8, SEQ ID NO: 9, and SEQ ID NO: 10. In other aspects, the antifungal peptide or polypeptide comprise essentially of an amino acid sequence selected from of SEQ ID NO: 3, SEQ ID NO: 4, SEQ ID NO: 5, SEQ ID NO: 6, SEQ ID NO: 7, SEQ ID NO: 8, SEQ ID NO: 9, and SEQ ID NO: 10. In further aspects, the antifungal peptide or polypeptide comprise essentially of an amino acid sequence selected from of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and SEQ ID NO: 12.

In other embodiments, the peptide or polypeptides are conjugated to a non-natural moiety, such as a fusion protein, a stabilizing or targeting peptide or moiety, a pore forming molecule (e.g., mellatin) or a known fungicidal molecule (e.g., Fluconazole, Amphotericin B). In some aspects, the peptide or polypeptides can be combined in a delivery vehicle with other known antifungal molecules (e.g., Fluconazole, Amphotericin B) or can be used in a combination therapy. In some aspects, the peptide or polypeptides comprise at least one D amino acid or comprises all D amino acids.

In other aspects, the peptide or polypeptide comprises a cysteine in the peptide, wherein the cysteine is positioned at the N-terminus, the C-terminus or in both of these positions. In further aspects, the peptide or polypeptide is circularized. In some embodiments, the peptide or polypeptide is no more than 10 amino acids long, 11 amino acids long, 12 amino acids long, or 13 amino acids long. In still other aspects, the peptide or polypeptide is not circularized and/or is not N-terminally modified.

In additional embodiments, there are provided compositions comprising a peptide or polypeptide of selected from of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and SEQ ID NO: 12 in a pharmaceutically acceptable carrier. In some aspects, the composition is frozen or lyophilized.

In other embodiments, there are provided polynucleotide molecules consisting or consisting essentially of a nucleic acid sequence encoding an amino acid sequence selected from of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and SEQ ID NO: 12.

In further embodiments, there are provided polynucleotide molecules consisting or consisting essentially of a nucleic acid sequence encoding an amino acid sequence selected from of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and SEQ ID NO: 12 and, in some aspects, the nucleic acid sequence encoding the peptide is operably linked to a promoter, such as in an expression cassette or expression vector.

In some embodiments, there are provided are host cells which contains an expression cassette or expression vector comprising a nucleic acid sequence encoding an amino acid sequence selected from of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and SEQ ID NO: 12. In some aspects, the host cell expresses a peptide having an amino acid sequence selected from of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and SEQ ID NO: 12. In further aspects, the host cell is that of a bacterium, yeast, mammal, human or plant.

In some embodiments, methods are provided for manufacturing a recombinant polypeptide comprising: (a) expressing a polynucleotide molecule described herein; and (b) isolating the polypeptide from the cell.

In other embodiments, there are provided methods of treating or preventing a fungal disease in a subject comprising administering an effective amount of an antifungal peptide or polypeptide or the described compositions alone or in combination with other antifungal therapies. In some aspects, the peptide, polypeptide or composition is administered via a route selected from oral, topical, vaginal, intranasal, intraperitoneal, parenteral, intravenous, intramuscular, subcutaneous, intrathecal, transcutaneous, nasopharyngeal, or via transmucosal absorption, such as by intravenous injection or catheter delivery.

In some embodiments, the fungal disease is associated with, caused by, or the result of organisms in the Candida, Aspergillus, Histoplasma, Cryptococcus, Coccidioides, Paracoccidioides, Blastomyces, Mucor, Rhizopus, Scedosporium, Pneumocystis, Penicillium, Fusarium, Tinea, Malassezia, Trichophyton, Microsporum, or Epidermophyton genera. In some embodiments, the methods of treating or preventing a fungal disease or disorder are applied to human or non-human mammalian subjects.

In alternative embodiments, compositions are provided comprising the described peptides or polypeptides are formulated for application to a surface or a consumable product. In some aspects, the composition is in the form of a paint or coating. In other aspects, the coating is an architectural coating, an industrial coating, or a specification coating. In additional aspects, the composition is part of a multicoat system. In further aspects, the composition comprises a binder, such as a thermoplastic binder, a thermosetting binder, or a combination thereof.

In another embodiments, methods are provided for inhibiting fungal growth on a surface or in a consumable product comprising contacting the composition with a surface or consumable product. In some aspects, the method of inhibiting fungal growth on a surface or in a consumable product comprises contacting the composition with a surface or consumable product. In further aspects, the consumable product is a foodstuff or a liquid. In a further embodiment, kits are provided comprising the antifungal peptides or compositions as described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1C: The sequence and structure of EntV. (FIG. 1A) The sequence of full-length, secreted EntV¹³⁶ (SEQ ID NO: 13). The second line is EntV⁶⁸ (SEQ ID NO: 14), the active form of the protein generated following cleavage. The highlighted sequences and their colors correspond to the alpha helices resolved in the X-ray structure shown in (FIG. 1B). (FIG. 1C) The structure and composition of the central α7 helix.

FIGS. 2A-2D: The α7 helix of EntV has antifungal activity. (FIG. 2A) and (FIG. 2C) show survival over time of C. elegans infected with C. albicans and exposed to 1 nM of the indicated fragments of EntV based on the structure in FIG. 1 (FIG. 2B) or versions of the ═7 helix (C). Random sequence=TNVGALWICGLAVIQAAQLS (SEQ ID NO: 1) and disrupted sequence=TNAAAAQQGGIICLLVVWLS (SEQ ID NO: 2). Statistical differences in survival were compared to animals in the exposure condition on the left of each legend by Mantel-Cox log rank analysis. An n of 60 animals was used and one representative trial is shown. Exact p values from top to bottom: (FIG. 2A) <0.0001, 0.1900, 0.0361, 0.0583, 0.0059, 0.0019. (FIG. 2C) <0.0001, 0.1666, <0.0001, <0.0001. Median survival and p values of all trials are shown in Table 1. (FIG. 2B) and (FIG. 2D) show adhesion of C. albicans to tissue-culture treated plates following incubation with different concentrations of the indicated peptides. “Neg” is a control nonadherent strain (Δefg1 Δcph1). Lines with error bars indicate the mean and the SD following normalization against the mean of the vehicle control group. Statistical significance in comparison to the DMSO control group was determined using one-way ANOVA followed by Dunnett's multiple comparison test. An n of three biological replicates, each with six technical replicates, was used. Exact p values from left to right are as follows: (FIG. 2B) 0.1065, <0.0001, 0.0071, 0.0001, 0.9998, 0.0026, 0.9997, 0.8961, 0.9998, 0.9875, 0.0002, (FIG. 2D) 0.0101, 0.1223, 0.9994, 0.8044, 0.8456, 0.9996, 0.9994, 0.993, 0.8957, <0.0001.

FIGS. 3A-3F: Shorter variants of α7 retain antifungal activity. (FIG. 3A), (FIG. 3C) and (FIG. 3E) show survival over time of C. elegans infected with C. albicans and exposed to 1 nM of the indicated α7 fragment as shown in (FIG. 3F; SEQ ID NO: 15). Statistical differences in survival were compared to animals in the exposure condition on the left of each legend by Mantel-Cox log rank analysis. An n of 60 was used and one representative trial is shown. Exact p values from top to bottom: (FIG. 3A) <0.0001, 0.7721, 0.4591, 0.5702, 0.3797, (FIG. 3C) <0.0001, 0.7958, 0.2951, 0.5476, 0.0002. (FIG. 3E) <0.0001, 0.0489, 0.5844, 0.0127, <0.0001. Median survival and p values of all trials are shown in Table 1. (FIG. 3B) and (FIG. 3D) show adhesion of C. albicans to tissue-culture treated plates following incubation with 10 nM of the α7 fragment. “Neg” is the background observed with a non-adherent strain (Δefg1Δcph1). Lines with error bars indicate the mean and the SD following normalization against the mean of the vehicle control group. Statistical significance in comparison to the DMSO control group was determined using one-way ANOVA followed by Dunnett's multiple comparison test. An n of four biological replicates, each with six technical replicates, was used. Exact p values from left to right: (FIG. 3B) 0.0003, 0.0210, 0.0001, (FIG. 3D) <0.0001, 0.6890, <0.0001.

FIGS. 4A-4F: The wild-type 12-mer is protective in a mouse model of oropharyngeal candidiasis. Amount of C. albicans DNA detected by qPCR, (FIG. 4A), and percentage of the tongue surface showing hyphal invasion, (FIG. 3B), in untreated animals compared to those given 100 nM EntV and 12aa α7 in their drinking water for 5 days following inoculation. (FIG. 4C) Representative pictures used to score hyphal invasion. Pink staining cells in the areas that are magnified, indicated by the black arrow heads, are C. albicans. The cells in the control sample show an elongated, hyphal morphotype more prevalently invading the tissue compared to the treated samples. (FIG. 4D) The amount of C. albicans DNA detected by qPCR in animals treated with 100 nM of different fragment lengths of the α7 peptide at day 5. (FIG. 4E) The amount of C. albicans DNA detected by qPCR in animals at day 3 or 5 following treatment with 100 nM of the 12-mer starting at day 0 or 3. (FIG. 4F) The amount of C. albicans DNA detected by qPCR in animals treated with different concentrations of the 12-mer in comparison to fluconazole at day 5. An n of 6-10 biologically independent animals was used with the exact number indicated by the number of data points. Averages were calculated and the error bars represent the SEM. Horizontal lines mark compared conditions. Significance was determined using one-way ANOVA followed by Tukey's multiple comparison test. Exact p values from top to bottom are as follows: (FIG. 4A) <0.0001, <0.0001, >0.9999, (FIG. 4B) <0.0001, <0.0001, 0.8811, (FIG. 4D) <0.0001, 0.0014, 0.0206, 0.0515, (FIG. 4E) <0.0001, 0.9688, 0.0030, 0.0009, (FIG. 4F) 0.8588, <0.0001, <0.0001, 0.0043, 0.0001, 0.1672, 0.0444, 0.9087, 0.0282.

FIGS. 5A-5C: The wild-type 12-mer is protective in models of venous catheter and systemic C. albicans infection. Intravenous catheters were implanted into the jugular veins of rats and inoculated with C. albicans with or without EntV peptides. After 24 h the catheters were removed CFUs were determined from one-half of each catheter (left panel) and scanning electron micrographs at 100× (left) or 1000× (right) from the other half of the catheters (right panels). (FIG. 5A) Treatment with EntV at 100 nM. (FIG. 5B) Treatment with the wild-type 12-mer at 20 nM. The dashed line is the detection limit of this assay (100 CFU). (FIG. 5C) C. albicans was incubated for 2 h in PBS with DMSO, EntV, or the 12-mer prior to inoculation via the lateral tail vein. When moribund, animals were humanely sacrificed. Survival was compared to the DMSO control by Mantel-Cox log rank analysis and the exact p values were <0.0001 and <0.0001.

FIGS. 6A-6D: EntV peptides have activity against many fungal species and strains. Survival over time of C. elegans infected with C. auris, (FIG. 6A), C. neoformans, (FIG. 6B), and C. albicans, (FIG. 6C), paired clinical isolates before (AzS: fluconazole-susceptability) and after gaining fluconazole resistance (AzR), (FIG. 6D). Peptides were added at 1 nM and fluconazole at 4 ug/ml. Statistical differences in survival were compared to animals to the DMSO vehicle control by Mantel-Cox log rank analysis. An n of 60 was used and one representative trial is shown. Exact p values from top to bottom: (FIG. 6A) <0.0001, <0.0001, (FIG. 6B) <0.0001, <0.0001, (FIG. 6C) 0.0013, 0.0004, 0.0035, (FIG. 6D) 0.0050, 0.0040, 0.5539.

FIGS. 7A-7B: The charged residues of EntV are located asymmetrically. (FIG. 7A) The outer surface of the N-terminal domain (NTD) of EntV¹³⁶ is negatively charged (red) whereas the outer surface of the C-terminal domain (CTD) is positively charged (blue). (FIG. 7B) The structure of Ent V⁶⁸ (α4-α7) modeled by removal of the NTD. The outer surface of α4-α6 is positively charged whereas the inner surface that largely faces α7 is hydrophobic/neutral.

FIG. 8: EntV fragments have no inhibitory activity against Lactobacillus sakei. L. sakei was cultured in Lactobacilli MRS broth overnight and diluted 1/10 into fresh medium to which EntV and EntV fragments were added at various concentrations. After 24 hours of growth at 30° C., the OD₆₂₅ readings were taken. Asterisks mark the samples in which a statistically significant was observed in comparison to the DMSO control.

FIGS. 9A-9C: Some substitutions of the charged residues of α7 affect antifungal activity. (FIG. 9A) and (FIG. 9C) show survival over time of C. elegans infected with C. albicans and exposed to 1 nM of the indicated peptides. The glutamines were changed to glutamates in α7-EE and to isoleucines in a7-II in (FIG. 9A). The cysteine of the wild-type 12-mer was changed to a serine or chemically alkylated in (FIG. 9C). Asterisks indicate the degree of statistical difference in survival compared to animals exposed to the wild-type versions of the peptides on the left side of each legend. The data are representative of two independent trials performed in duplicate for an n of 60. Median survival and p values of all trials are shown in Table 1. (FIG. 9B) Adhesion of C. albicans to tissue-culture treated plates following incubation with indicated concentrations of § 7 fragments. “Neg” is a control non-adherent strain (Δefg1 Δcph1). Results represent data collected across three biological replicates and asterisks indicate degree of statistical difference in comparison to the DMSO control. Note that the α7-II peptide was statistically significant at the lower concentrations due to slightly better adhesion than the control.

FIGS. 10A-10F: Shorter variants of α7 retain adhesion activity down to 10aa. Adhesion of C. albicans to tissue-culture treated plates following incubation with the indicated concentrations of EntV (FIG. 10A) and α7 variants of 16aa (FIG. 10B), 12aa (FIG. 10C), 11aa (FIG. 10D), 10aa (FIG. 10E) and 9aa (FIG. 10F). Lines with error bars indicate the mean and the SD following normalization against the mean of the vehicle control group. Significance was determined using one-way ANOVA followed by Dunnett's multiple comparison test. Lines with error bars indicate the mean and the SD following normalization against the mean of the vehicle control group. Statistical significance in comparison to the DMSO control group was determined using one-way ANOVA followed by Dunnett's multiple comparison test. An n of three (a-b) or four (c-f) biological replicates, each with six technical replicates, was used. Exact p values from left to right are as follows: (FIG. 10A) 0.1407, 0.0170, 0.9998, 0.9997, 0.9999, 0.9998, 0.0009, (FIG. 10B) 0.0002, 0.0003, 0.8038, 0.9502, 0.7657, 0.0003, (FIG. 10C) <0.00001, <0.0001, 0.0004, 0.9973, 0.9998, 0.9999, <0.0001, (FIG. 10D) 0.0008, 0.0028, 0.9523, 0.9930, 0.9594, 0.9997, <0.0001, (FIG. 10E) <0.0001, <0.0001, 0.9336, 0.8043, 0.9999, 0.9998, <0.0001, (FIG. 10F) 0.9627, 009518, 0.9999, >0.9999, 0.9840, 0.9995, and <0.0001.

FIGS. 11A-11G: The wild-type 12-mer (SEQ ID NO: 4) is protective in a mouse model of oropharyngeal candidiasis. (FIG. 11A) The percentage of the tongue surface showing hyphal invasion in animals treated with 100 nM of different peptide fragment lengths of the α7 peptide. (FIG. 11B) The percentage of the tongue surface showing hyphal invasion in animals at day 3 or 5 post-inoculation following treatment with 100 nM of the 12-mer (SEQ ID NO: 4) starting at day 0 or 3. (FIG. 11C) The percentage of the tongue surface showing hyphal invasion in animals treated with different concentrations of the 12-mer in comparison to fluconazole. Amount of C. albicans DNA detected by qPCR, (FIG. 11D), and percentage of the tongue surface showing hyphal invasion, (FIG. 11E), in animals treated with different concentrations of the 12-mer. Amount of C. albicans DNA detected by qPCR, (FIG. 11F), and percentage of the tongue surface showing hyphal invasion, (FIG. 11G), disaggregated by sex in animals treated with 100 nM of EntV. An n of 5-8 biologically independent animals was used with each animal shown as a separate point in the figure panels. Averages were calculated and the error bars represent the SEM. Horizontal lines mark compared conditions and the exact p values are given. Significance was determined using one-way ANOVA followed by Tukey's multiple comparison test. Exact p values from top to bottom are as follows: (FIG. 11A) 0.0003, 0.0019, 0.0065, 0.9999, (FIG. 11B) <0.0001, 0.0250, 0.0250, 0.0240, 0.9935, (FIG. 11C) 0.0304, <0.0001, <0.0001, 0.0002, 0.0228, 0.9999, 0.3923, 0.5257, 0.5011, (FIG. 11D) 0.0864, 0.0273, 0.9517, 0.0033, <0.0001, 0.0009, (FIG. 11E) 0.0152, 0.0383, 0.0015, 0.9663, 0.6514, 0.3885, (FIG. 11F) <0.0001, <0.0001, 0.0050, 0.0056, 0.1110, (FIG. 11G) <0.0001, <0.0001, 0.0135, 0.0011, 0.1550.

FIGS. 12A-12F: Mutational analysis of the 12aa variant of EntV. (FIG. 12A; SEQ ID NOS: 4, 6, and 16-21) and (FIG. 12B; SEQ ID NOS: 4, 5, and 22-27) show the sequence alignments of the parent 10aa and 11aa variants, respectively, against the 12aa sequence, as well as the positional mutations for the alanine scan. (FIG. 12C) and (FIG. 12D) show survival over time of C. elegans infected with C. albicans and exposed to 1 nM of the 10aa and 11aa peptides, respectively as described in (A) and (B). Statistical differences in survival were compared to animals in the exposure condition on the right of each legend (10aa-WT and 11aa-WT) by Mantel-Cox log rank analysis. An n of 30 animals was used and one representative trial is shown. (FIG. 12E; SEQ ID NOS: 4, 28, and 29) shows the sequence alignments of the parent 12aa peptide and two variants with directed mutations to increase the overall hydrophobicity of the peptide. In V3, the native alanine residues were swapped with valine residues; in L3, the native alanine residues were swapped with leucine residues. (FIG. 12F) show survival over time of C. elegans infected with C. albicans and exposed to 1 nM of the peptides generated in (E). For all statistical tests, p values<0.05 were considered statistically significant. *p<0.05, **p<0.01, ***p<0.001, ****p<0.0001.

FIGS. 13A-13C: Modifications to increase the stability of the 12aa peptide do not ablate antifungal activity. (FIG. 13A) Peptides were exposed to 10% human serum and their stability over time tracked using HPLC purification to measure the amount of remaining peptide. A modified chi-squared curve analysis was used to compare the stability of the modified peptides to the native L-form 12aa peptide. The error bars represent the (SEM) (FIG. 13B) and (FIG. 13C) show survival over time of C. elegans infected with C. albicans and exposed to 1 nM of the indicated peptides. Statistical differences in survival were compared to animals in the exposure condition on the right of each legend (12aa-WT) by Mantel-Cox log rank analysis. An n of 30 animals was used per experimental group and one representative trial is shown. Median survival and p values of all trails are presented in Table 1. For all statistical tests, p values<0.05 were considered statistically significant. *p<0.05, **p<0.01, ***p<0.001, ****p<0.0001.

FIGS. 14A-14C: Combinatorial peptide library generation and high-throughput screening identifies 5 gain-of-function variants. (FIG. 14A) The synthesis map outlining the positional flexible residues and the different amino acids added for combinatorial chemistry during solid-phase peptide synthesis (SPPS) to generate the library of 486 new sequences (SEQ ID NO: 5). (FIG. 14B) An orthogonal plot of the relative efficacy of the library-generated peptides in protecting C. elegans infected with C. albicans as measured by the high-throughput Sytox Orange assay (x-axis) and in reducing C. albicans biofilm formation (y-axis). Each peptide was tested in both experiments with 3 controls: the untreated DMSO control, the 10aa parent sequence, and the 12aa benchmark peptide for antifungal activity. (FIG. 14C) shows the sequences of the five lead candidates, starred in (B), compared to the parent 10aa wild-type sequence (SEQ ID NOS: 6, 8-11).

FIGS. 15A-15D: Secondary testing of gain-of-function variants from screen. (FIG. 15A) Survival over time of C. elegans infected with C. albicans and exposed to 1 nM of the indicated peptides. Statistical differences in survival were compared to animals in the exposure condition on the right of the legend (12aa-WT) by Mantel-Cox log rank analysis. An n of 30 animals was used and one representative trial is shown. (FIG. 15B) Percent survival as measured by the high-throughput Sytox Orange assay following five days of infection with C. albicans is shown. Means were calculated with the error bars representing the SEM. Statistical differences in survival were compared to animals in the untreated condition (DMSO) by one-way ANOVA followed by Dunnett's multiple comparison test. One representative trial is shown with two biological replicates presented in FIG. 18. An n of 30 animals was used per experimental group. (FIG. 15C) Adhesion of C. albicans to tissue-culture treated plates following incubation with 10 nM of the indicated peptides is shown. A non-adherent strain (Δefg1/Δcph1) was used as a control. Means were calculated with the error bars representing the SEM. Statistical significance in comparison to the DMSO control group was determined using one-way ANOVA followed by Tukey's multiple comparison test. An n of three biological replicates, each with four technical replicates, was used. (FIG. 15D) Percent survival as measured by the high-throughput Sytox Orange assay following five days of infection with C. neoformans is shown. Means were calculated with the error bars representing the SEM. Statistical differences in survival were compared to animals in the untreated condition (DMSO) by one-way ANOVA followed by Dunnett's multiple comparison test. One representative trial is shown with three biological replicates presented in FIG. 19. An n of 30 animals was used per experimental group. For all statistical tests, p values<0.05 were considered statistically significant. *p<0.05, **p<0.01, ***p<0.001, ****p<0.0001.

FIGS. 16A-16C: The 10aa variants P1 and P4 demonstrate enhanced protection in a mouse model of oropharyngeal candidiasis compared to the 10aa parent. (FIG. 16A) reports the amount of C. albicans DNA detected by qPCR and (FIG. 16B) measured the percentage of the tongue surface showing hyphal invasion in animals given 100 nM of the indicated peptides or the vehicle control (DMSO) in their drinking water for 5 days following inoculation. Means were calculated with the error bars representing the SEM. As indicated by the lines, statistical differences in survival were compared by one-way ANOVA followed by Tukey's multiple comparison test. For all statistical tests, p values<0.05 were considered statistically significant. *p<0.05, **p<0.01, ***p<0.001, ****p<0.0001. (FIG. 16C) shows representative images used to score hyphal invasion of the tongue tissue. Pink staining cells in the areas that are magnified, indicated by the black arrows, are C. albicans. The cells in the control DMSO sample show the hyphal morphotype of C. albicans are more prevalent than the peptide-treated tissue samples.

FIGS. 17A-17D: The stability-enhanced 12aa variants and the 10aa P4 variant have antifungal activity in a mouse model of systemic candidiasis. C. albicans was inoculated by tail vein injection following a 2-hour preincubation with 100 nM of the indicated peptides. Fungal organ burden was assessed by measuring C. albicans DNA by qPCR in the kidneys (FIG. 17A) and liver (FIG. 17C). Means were calculated with the error bars representing the SEM. Statistical differences were compared by one-way ANOVA followed by Tukey's multiple comparison test for all samples with only statistically significant differences indicated with lines. For all statistical tests, p values<0.05 were considered statistically significant. *p<0.05, **p<0.01, ***p<0.001, ****p<0.0001. (FIG. 17B) and (FIG. 17D) show representative images of the kidney and liver tissue, respectively, at Day 2 and Day 5. The areas indicated by the black arrows show C. albicans.

FIG. 18: Additional biological replicates of the C. albicans infection assay with C. elegans using Sytox Orange. Two additional biological replicates of the experiment presented in FIG. 15B were performed to assess percent survival as measured by the high-throughput Sytox Orange assay following five days of infection with C. albicans and the indicated treatments. Means were calculated with the error bars representing the SEM. Statistical differences in survival were compared to animals in the untreated condition (DMSO) by one-way ANOVA followed by Dunnett's multiple comparison test. An n of 30 animals was used per experimental group. For all statistical tests, p values<0.05 were considered statistically significant. *p<0.05, **p<0.01, ***p<0.001, ****p<0.0001.

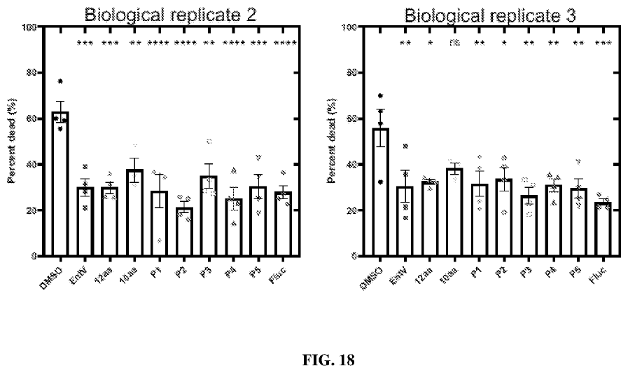

FIG. 19: Additional biological replicates of the C. neoformans infection assay with C. elegans using Sytox Orange. Three additional biological replicates of the experiment presented in FIG. 15D were performed to assess percent survival as measured by the high-throughput Sytox Orange assay following five days of infection with C. albicans and the indicated treatments. Means were calculated with the error bars representing the SEM. Statistical differences in survival were compared to animals in the untreated condition (DMSO) by one-way ANOVA followed by Dunnett's multiple comparison test. An n of 30 animals was used per experimental group. For all statistical tests, p values<0.05 were considered statistically significant. *p<0.05, **p<0.01, ***p<0.001, ****p<0.0001.

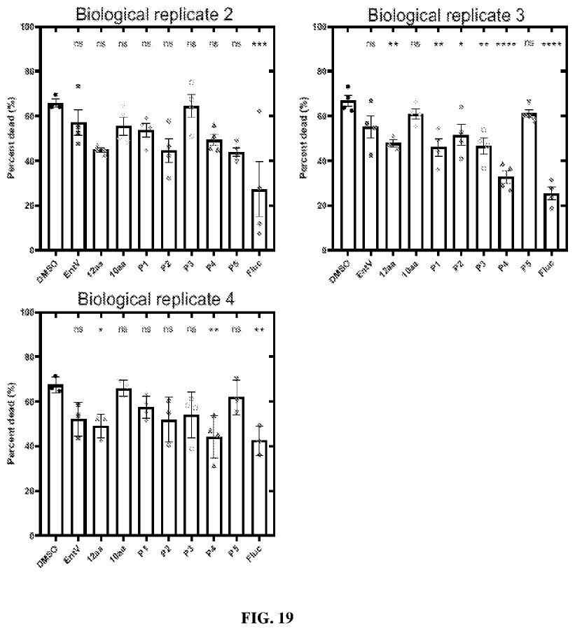

### DETAILED DESCRIPTION

### A. DEFINITIONS

In this disclosure, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements and components comprising one unit and elements or components that comprise more than one unit unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073.

In calculating percent identity, the sequences being compared are typically aligned in a way that gives the largest match between the sequences. One example of a computer program that can be used to determine percent identity is the GCG program package, which includes GAP (Devereux et al., 1984, Nucl. Acid Res. 12:387; Genetics Computer Group, University of Wisconsin, Madison, Wis.). The computer algorithm GAP is used to align the two polypeptides or polynucleotides for which the percent sequence identity is to be determined. The sequences are aligned for optimal matching of their respective amino acid or nucleotide (the “matched span”, as determined by the algorithm). A gap opening penalty (which is calculated as 3× the average diagonal, wherein the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. In certain embodiments, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. U.S.A. 89:10915-10919 for the BLOSUM 62 comparison matrix) is also used by the algorithm.

Examples of parameters that can be employed in determining percent identity for polypeptides or nucleotide sequences using the GAP program can be found in Needleman et al., 1970, J. Mol. Biol. 48:443-453.

Certain alignment schemes for aligning two amino acid sequences may result in matching of only a short region of the two sequences, and this small aligned region may have very high sequence identity even though there is no significant relationship between the two full-length sequences. Accordingly, the selected alignment method (GAP program) can be adjusted if so desired to result in an alignment that spans at 50 or fewer contiguous amino acids of the target peptide or polypeptide.

The term “link” as used herein refers to the association via intramolecular interaction, e.g., covalent bonds, metallic bonds, and/or ionic bonding, or inter-molecular interaction, e.g., hydrogen bond or noncovalent bonds.

The term “operably linked” refers to an arrangement of elements wherein the components so described are configured so as to perform their usual function. Thus, a given signal peptide that is operably linked to a polypeptide directs the secretion of the polypeptide from a cell. In the case of a promoter, a promoter that is operably linked to a coding sequence will direct the expression of the coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

The term “polynucleotide” or “nucleic acid” includes both single-stranded and double-stranded nucleotide polymers. The nucleotides comprising the polynucleotide can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate.

As used herein, “antifungal” or “antifungal activity” refer to those molecules, macromolecules, compounds, chemicals, peptides, polypeptides and alike, that have antifungal activities such as, but not limited to fungicidal activity, fungistatic activity and anti-virulence activity. The terms “polypeptide” or “peptide” means a macromolecule having the amino acid sequence of an antifungal protein, that is, a protein produced by a naturally-occurring and non-recombinant cell; or it is produced by a genetically-engineered or recombinant cell, and comprise molecules having the amino acid sequence of the a protein, or molecules having deletions from, additions to, and/or substitutions of one or more amino acids of the an antifungal protein sequence. The term also includes amino acid polymers in which one or more amino acids are chemical analogs of a corresponding naturally occurring amino acid and polymers. The terms “peptide” “polypeptide” and “protein” specifically encompass antifungal sequences that have deletions from, additions to, and/or substitutions of one or more amino acids. Such fragments can also contain modified amino acids as compared with the native protein. In certain embodiments, the peptide fragments are 68 amino acids in length, but preferably they are from 12 amino acids to 5 amino acids in length. For example, fragments can be 5, 6, 7, 8, 9, 10, 11 and 12 amino acids in length or longer.

As used herein, the phrase “pharmaceutically acceptable” is employed herein to refer to those compounds, materials, compositions, and/or dosage forms which are, within the scope of sound medical judgment, suitable for use in contact with the tissues of human beings or, as the case may be, an animal without excessive toxicity, irritation, allergic response, or other problem or complication, commensurate with a reasonable benefit/risk ratio. The pharmaceutically acceptable carriers useful in this disclosure are conventional. Remington's Pharmaceutical Sciences, by E. W. Martin, Mack Publishing Co., Easton, PA, 15th Edition (1975), describes compositions and formulations suitable for pharmaceutical delivery of the fusion proteins herein disclosed. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Thus, the disclosure encompasses the salt or pro-drug of a peptide or peptide variant of the disclosure. The peptide of the disclosure may be administered in the form of pharmaceutically acceptable salt. The pharmaceutically acceptable salts of the present disclosure can be synthesized from the parent peptide which contains a basic or acidic moiety by conventional chemical methods. Generally, such salts can be prepared by reacting the free acid or base forms of the peptide with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., US, 1985, p. 1418, the disclosure of which is hereby incorporated by reference; see also Stahl et al, Eds, “Handbook of Pharmaceutical Salts Properties Selection and Use”, Verlag Helvetica Chimica Acta and Wiley-VCH, 2002.

The disclosure thus includes pharmaceutically acceptable salts of the peptide of the disclosure wherein the parent compound is modified by making acid or base salts thereof for example the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, malonate, methanesulfonate, 2-naphthalenesulfonate, nicotinate, oxalate, palmoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glutamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

Salts of carboxyl groups of a peptide or peptide variant of the disclosure may be prepared in the usual manner by contacting the peptide with one or more equivalents of a desired base such as, for example, a metallic hydroxide base, e.g., sodium hydroxide; a metal carbonate or bicarbonate such as, for example, sodium carbonate or bicarbonate; or an amine base such as, for example, triethylamine, triethanolamine and the like.

The disclosure includes prodrugs for the active pharmaceutical species of the described peptide, for example in which one or more functional groups are protected or derivatized but can be converted in vivo to the functional group, as in the case of esters of carboxylic acids convertible in vivo to the free acid, or in the case of protected amines, to the free amino group. The term “prodrug,” as used herein, represents in particular structures which are rapidly transformed in vivo to the parent structure, for example, by hydrolysis in blood.

As used herein, the term “subject” refers to a human or any non-human animal (e.g., mouse, rat, rabbit, dog, cat, cattle, swine, sheep, horse or primate). A human includes pre-and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of a fungal disease or disorder.

The term “therapeutically effective amount” or “effective dosage” as used herein refers to the dosage or concentration of a drug effective to treat a fungal disease or condition. For example, with regard to the use of the antifungal peptides disclosed herein to fungal infections, a therapeutically effective amount is the dosage or concentration of the anti-fungal peptide capable of reducing the fungal infection inhibiting growth or proliferation of fungi, preventing or delaying the development of a fungal infection, or some combination thereof.

“Treating” or “treatment” of a condition as used herein includes preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof. The treatment step may comprise topical application to the susceptible organism such as topical creams and ointments in the case of animals, or as sprays, fogs, mists, powders and the like in the case of plants. In other instances, it will be preferred to treat the susceptible organism with oral, nasal, gavage, enema, suppository or other internal application. In others instances, the treatment step will comprise application by injection

As used herein, a “vector” refers to a nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

The peptides described herein are useful, inter alia, as an antifungal peptide. The term, “antifungal peptide” can be used herein to define any peptide that has fungicidal and/or fungistatic activity and encompasses, non-exclusively, any peptide described as having anti-fungal properties. In a preferred aspect, the disclosure provides the use of a peptide according to the disclosure in the manufacture of a medicament for treating a fungal infection in a mammal.

Recombinant or synthetic antifungal peptide compositions that inhibit fungal growth which have both medical and non-medical utility and application, including but not limited to treat, prevent or otherwise limit fungal damage to agricultural and horticultural crops, particularly to seeds, seedlings and agricultural commodities, including mature plants such as trees. In addition, there is growing concern regarding the widespread use of horticultural antifungals, for example, with azole-family antifungal agents and the development of acquired drug resistance in pathogens that can also infect humans in response to environmental exposure resulting from agricultural use. This concern is amplified because some mechanisms of acquired drug resistance also reduce susceptibility to other chemical classes of antifungal agents.

Certain of the compositions of the disclosure may be used to treat non-plant fungal diseases, such as those of animals including man. The compositions described herein are especially useful where conditions are conducive to fungal disease development and where control of fungal growth is preferably accomplished with compositions which are not toxic to non-fungal cells. This includes but is not limited to fungal growth on indoor and outdoor surfaces, which are a major environmental concern that affects the home, work and recreational environments.

After obtaining EntV structural data, the inventors determined that antifungal activity was isolated to a single α-helix that can be reduced to 10-12 amino acids with full to partial activity in multiple in vitro, ex vivo, and in animal virulence models. Based on these findings the inventors further identified and characterized additional peptides with antifungal properties.

A 12 amino acid fragment of EntV was determined to retain full antifungal activity, with partial activity observed down to 10 amino acids. The 12-mer displayed excellent efficacy against C. albicans in mouse models of OPC, where it both prevented infection and treated established infections. It also protected against systemic infection and prevented biofilm formation on implanted venous catheters.

Based on these findings the inventors executed mutational analysis and combinatorial library screening to generate and characterize additional peptides with antifungal properties. Several of the peptides identified were determined to prevent infection and in the oral candidiasis model to effectively treat infection. Several of these peptides reduced fungal biofilm adhesion in vitro as well as in implanted intravenous catheters. Finally, some of these peptides were effective in a systematic model that mimics deadly human bloodstream infections.

No apparent toxicity was seen in the mouse model, and previous studies observed no toxicity when EntV was incubated with human and mouse cells. It was also determined that the shorter wild-type 12-mer no longer had bacteriocin activity, indicating that the antifungal and antibacterial activities can be isolated and reducing the chance for off-target effects.

In additional aspects, the antifungal peptide compositions may be further modified by one or more other amino substitutions while maintaining their biological activity. For example, amino acid substitutions can be made at one or more positions wherein the substitution is for an amino acid having a similar hydrophilicity. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Thus often such conservative substitution can be made and will likely only have minor effects on their activity. As detailed in U.S. Pat. No. 4,554,101,the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (0.5); histidine−0.5); cysteine (−1.0); methionine (−1.3); valine (−1.5); leucine (−1.8); isoleucine (−1.8); tyrosine (−2.3); phenylalanine (−32.5); tryptophan (−3.4). These values can be used as a guide and thus substitution of amino acids whose hydrophilicity values are within ±2 are preferred, those that are within ±1 are particularly preferred, and those within ±0.5 are even more particularly preferred. Thus, amino acid sequences of the antifungal peptide described herein may be modified by the substitution of an amino acid, for different, but homologous amino acid with a similar hydrophilicity value. Amino acids with hydrophilicities within +/−1.0, or +/−0.5 points are considered homologous. Furthermore, it is envisioned that proline rich peptide segments of EntV, such as of the antifungal peptides may be modified by amino acid deletions, substitutions, additions or insertions while retaining its biological activity.

### B. PRODUCTION OF ANTIFUNGAL PEPTIDES

Large-scale synthesis of peptides in solution. The solution-phase synthesis allows easy planning with respect to group protection strategy, fragment selection and methods of fragment coupling to minimize racemization. The intermediates can sometimes be isolated simply by crystallization techniques, which may eliminate the need for purification by column chromatography and therefore improve the scale-up potential. The quality of simultaneously produced fragments can be easily controlled at each step.

Large scale solid-phase synthesis of peptides. The cost of the more advanced polymers for solid-phase synthesis is usually high. Some of the supports are not available in bulk. However, their properties play an important role in the accessibility of anchored peptide and release of the peptide from the resin in a fully protected, deprotected or modified form. The transition from laboratory to manufacturing scale of solid-phase peptide synthesis (SPPS) is clearly advantageous due to the fact that the entire synthetic process could be easily automated, and the efficiency of the synthetic steps could be monitored and optimized. The production scale activating processes are well known and environmentally harmless. In addition, SPPS allows direct recovery and recycling of excess of amino acid building blocks from the waste filtrates at production scale.

While the antifungal peptides described herein can be chemically synthesized (e.g., see Creighton, 1983), they may advantageously be produced by recombinant DNA technology using techniques well known in the art for expressing nucleic acids. Such methods can be used to construct expression vectors containing the nucleotide sequences that encode the amino acid sequences of the antifungal peptides described herein and appropriate transcriptional and translational control signals. These methods include, for example, in vitro recombinant DNA techniques, synthetic techniques, and in vivo genetic recombination. See, for example, the techniques described in Sambrook et al., 1989, supra, and Ausubel et al., 1989, supra. Alternatively, RNA capable of encoding the nucleotide sequence of the antifungal peptide sequences may be chemically synthesized using, for example, synthesizers. See, for example, the techniques described in “Oligonucleotide Synthesis”, 1984, Gait, M. J., ed., IRL Press, Oxford, which is incorporated by reference herein in its entirety.

A variety of host-expression vector systems can be utilized to express the nucleotide sequences that encode the amino acid sequences of the antifungal peptides described herein as embodiments of the disclosure. Where the antifungal peptide is a soluble derivative (e.g., with a deleted TMD), the polypeptide can be recovered from the culture, i.e., from the host cell in cases where the antifungal peptide is not secreted, and from the culture media in cases where the antifungal peptide is secreted by the cells. However, the expression systems also encompass engineered host cells that express the antifungal peptides or functional equivalents in situ, i.e., anchored in the cell membrane. Purification or enrichment of the antifungal peptides from such expression systems can be accomplished using appropriate detergents and lipid micelles and methods well known to those skilled in the art. However, such engineered host cells themselves can be used in situations where it is important not only to retain the structural and functional characteristics of the antifungal peptides, but to assess biological activity, e.g., in drug screening assays.

For long-term, high-yield production of recombinant proteins, stable expression is preferred. For example, cell lines which stably express the antifungal peptide sequences can be engineered, for example, as described in SEQ ID NOs: 3-10 and in the examples below. Rather than using expression vectors which contain viral origins of replication, host cells can be transformed with DNA controlled by appropriate expression control elements (e.g., promoter, enhancer sequences, transcription terminators, polyadenylation sites, etc.), and a selectable marker. Following the introduction of foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express antifungal peptides. Such engineered cell lines may be particularly useful in screening and evaluation of compounds that affect the production of the antifungal peptides. A number of selection systems can be used, including but not limited to the herpes simplex virus thymidine kinase (Wigler, et al., 1977), hypoxanthine-guanine phosphoribosyltransferase (Szybalska & Szybalski, 1962), and adenine phosphoribosyltransferase (Lowy, et al., 1980) genes can be employed in tk-, hgprt- or aprt- cells, respectively. Also, anti-metabolite resistance can be used as the basis of selection for the following genes: dhfr, which confers resistance to methotrexate (Wigler, et al., 1980; O'Hare, et al., 1981); gpt, which confers resistance to mycophenolic acid (Mulligan & Berg, 1981); neo, which confers resistance to the aminoglycoside G-418 (Colberre-Garapin, et al., 1981); and hygro, which confers resistance to hygromycin (Santerre, et al., 1984).

The expression systems that can be used for purposes of the embodiments include but are not limited to microorganisms such as bacteria (e.g., E. coli, B. subtilis) transformed with recombinant bacteriophage DNA, plasmid DNA or cosmid DNA expression vectors containing antifungal peptide nucleotide sequences; yeast (e.g., Saccharomyces, Pichia) transformed with recombinant yeast expression vectors containing the nucleotide sequences that encode the antifungal peptides; insect cell systems infected with recombinant virus expression vectors (e.g., baculovirus) containing the nucleotide sequences that encode the amino acid sequences of the antifungal peptides described herein; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing nucleotide sequences that encode the amino acid sequences of the antifungal peptides; or mammalian cell systems (e.g., COS, CHO, BHK, 293, 3T3) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors may be advantageously selected depending upon the use intended for the antifungal peptide being expressed. For example, when a large quantity of such a protein is to be produced, for the generation of pharmaceutical compositions of the antifungal peptides described herein, for example, vectors which direct the expression of high levels of fusion protein products that are readily purified may be desirable. Such vectors include, but are not limited, to the E. coli expression vector pUR278 (Ruther et al., 1983), in which the antifungal peptide coding sequence may be ligated individually into the vector in frame with the lacZ coding region so that a fusion protein is produced; pIN vectors (Inouye & Inouye, 1985; Van Heeke & Schuster, 1989); and the like. pGEX vectors may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhidrosis virus (AcNPV) is used as a vector to express foreign sequences. The virus grows in Spodoptera frugiperda cells. The nucleotide sequences that encode the amino acid sequences of the antifungal peptides described herein may be cloned individually into non-essential regions (for example the polyhedrin gene) of the virus and placed under control of an AcNPV promoter (for example the polyhedrin promoter). Successful insertion the nucleotide sequences that encode the amino acid sequences of the antifungal peptides described herein will result in inactivation of the polyhedrin gene and production of non-occluded recombinant virus, (i.e., virus lacking the proteinaceous coat coded for by the polyhedrin gene). These recombinant viruses are then used to infect Spodoptera frugiperda cells in which the inserted polynucleotide is expressed (e.g., see Smith et al., 1983 and U.S. Pat. No. 4,215,051).

In mammalian host cells, a number of viral-based expression systems can be utilized. In cases where an adenovirus is used as an expression vector, the nucleotide sequences that encode the amino acid sequences of the antifungal peptides of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the nucleotide sequences that encode the amino acid sequences of the antifungal peptides described in infected hosts (e.g., See Logan & Shenk, 1984). Specific initiation signals may also be important for efficient translation of inserted antifungal peptide encoding nucleotide sequences. These signals include the ATG initiation codon and adjacent sequences. In cases where nucleotide sequences that encode the amino acid sequences of the antifungal peptides or cDNA, including its own initiation codon and adjacent sequences, is inserted into the appropriate expression vector, no additional translational control signals may be needed. However, in cases where only a portion of the nucleotide sequences that encode the amino acid sequences of the antifungal peptides is inserted, exogenous translational control signals, including, perhaps, the ATG initiation codon, must be provided. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (See Bitter, et al.,1987).

In yeast, a number of vectors containing constitutive or inducible promoters may be used. For a review, see Current Protocols in Molecular Biology, 1988; Grant, et al., 1987; Wu & Grossman, 1987; Bitter, 1987; and “The Molecular Biology of the Yeast Saccharomyces”, 1982.

In cases where plant expression vectors are used, the expression of the coding sequence may be driven by any of a number of promoters. For example, viral promoters such as the 35S RNA and 19S RNA promoters of CaMV (Brisson et al., 1984), or the coat protein promoter of TMV (Takamatsu et al., 1987) may be used; alternatively, plant promoters such as the small subunit of RUBISCO (Coruzzi et al., 1984; Broglie et al., 1984); or heat shock promoters, e.g., soybean hsp17.5-E or hsp17.3-B (Gurley et al., 1986) may be used. These constructs can be introduced into plant cells using Ti plasmids, Ri plasmids, plant virus vectors, direct DNA transformation, microinjection, electroporation, etc. For reviews of such techniques see, for example, Methods for Plant Molecular Biology 1988; and Grierson & Corey, 1988.

### C. NON-MEDICAL APPLICATIONS OF ANTIFUNGAL PEPTIDES

Fungal growth on indoor and outdoor surfaces is a major environmental concern today affecting home, work and recreational environments. Not only can fungus (e.g., mold, mildew) be unsightly on exposed surfaces, it can destroy wood, fiber and other materials if left untreated, causing severe damage to buildings and other structures and equipment. Paints and paint films or coatings are known to be vulnerable to mold contamination due to the presence of common organic components that act as cellulosic thickeners, surfactants and defoamers, and which can also serve as a source of food for fungus cells. Some of these components are casein, acrylic, polyvinyl and other carbon polymers. For example, latex is a water-dispersed binder comprising a carbon polymer. Inside the paint can, certain fungi (e.g., yeasts) can convert enough carbon-containing food sources to CO₂ to swell or even explode the can. Fungi can also discolor and reduce the viscosity of the paint and produce foul odors. Both in-can preservation of paints and protection of the end use paint films, and the surfaces they cover, from mold, mildew and yeasts is necessary. To combat fungi, a variety of coating materials may be formulated which include organic or inorganic chemicals to discourage or prevent the growth of mildew on the paint film. Ideally, these chemical fungicides or mildewcides slowly leach out of the paint to the surface and maintain their inhibitory properties for the life of the paint film, causing little or no harm to the environment.

In some embodiments, the peptide compositions described herein overcome some of the disadvantages of previous antifungal or fungus-resistant paints, coatings and other compositions such as elastomers, textile finishes, adhesives, and sealants. By combining a natural, synthetic or recombinant bacteriostatic and/or antifungal polypeptide or peptide with a paint or other coating material to provide a coated surface with sustainable antifungal activity that protects the recipient surface from fungal infestation and defacement, and which also provides fungus resistance to the composition itself.

In some embodiments, new antifungal and antibacterial paints, coatings, films and other compositions are provided which contain one or more bioactive peptides, polypeptides and/or proteins as antifungal agents. Methods of using the antifungal and antibacterial additives and compositions for treating existing fungal or bacterial colonies and/or for deterring or preventing fungal or bacterial infestations and inhibiting cell growth or proliferation on a variety of inanimate objects such as interior and exterior architectural surfaces and building materials to prevent or deter or lessen the infestation and growth of fungus or bacterium. At the same time, the associated discoloration, disfiguration and/or degradation of the supporting substrate or surface can also be avoided or reduced.

The compositions of the disclosure are especially useful on surfaces where conditions are conducive to deposition and development of fungus or bacteria, and where control of fungal or bacterial growth is preferably accomplished with compositions which are not toxic to humans, pets and other animals or harmful to the environment.

The compositions and methods of the present disclosure overcome some of the disadvantages of previous antifungal or fungus-resistant paints, coatings and other compositions such as elastomers, textile finishes, adhesives, and sealants. In some embodiments, a combination of a natural, synthetic or recombinant antifungal peptide, polypeptide or peptides such as but not limited to EntV and the wild-type 12-mer (SEQ ID NO: 4) and includes (SEQ ID NO: 3-10) with a paint or other coating material to provide a coated surface with sustainable antifungal activity that protects the recipient surface from fungal infestation and defacement, and which also provides fungus resistance to the composition itself. Accordingly, new antifungal and antibacterial paints, coatings, films and other compositions comprising the peptides and proteins described herein are provided which contain one or more bioactive peptides, polypeptides and/or proteins as antifungal and antibacterial proteins and peptides.

A further embodiment comprises using a film or coat comprising the coating composition, or the composition itself, to protect an object or material selected from the group consisting of wood, paint, adhesive, glue, paper, textile, leather, plastic, cardboard, caulking, from infestation and growth of a fungus.

A preferred coating material comprises a paint or coating. In some embodiments the paint or coating is applied prophylactically over a “clean” surface that is not contaminated by fungal spores. In other embodiments the paint or coating is applied to a surface already contaminated by fungal spores or growing fungus.

Paints and other conventional protective or decorative coating materials typically contain polymeric substances such as casein, acrylic, polyvinyl and carbon polymers (e.g., binders) which can serve as nutrients for fungal cells. Not only can these nutrient substances support the growth of fungus on paint films or coated surfaces, fungus can also grow inside cans of liquid paints and coating compositions during storage. It was, therefore, an unexpected result that the addition of certain synthetic peptides, including but not limited to EntV and the wild-type 12-mer (SEQ ID NO: 4) and includes (SEQ ID NOS: 3-10), when added to a range of conventional paint and coating materials, render those compositions resistant to fungal infestation and growth. In some embodiments, such additives worked alone or in conjunction with existing biocides in a coating.

One or more of the antifungal peptides or peptide compositions, is mixed with a base paint or other coating, which may be any suitable commercially available product, a wide variety of which are well known in the art. Preferably the base composition is free of chemicals and other additives that are toxic to humans or animals, and/or that fail to comply with applicable environmental safety rules or guidelines. In some instances, it may be preferred to custom blend a paint or coating mixture using any combination of various naturally occurring and synthetic components and additives that are known in the art. Coating components generally include a binder, a liquid component, a colorizing agent, one or more additive, or a combination of any of those. A coating typically comprises a material often referred to as a “binder,” which is the primary material in a coating capable of producing a film. In most embodiments, a coating will comprise a liquid component (e.g., a solvent, a diluent, a thinner), which often confers and/or alters the coating's rheological properties (e.g., viscosity) to ease the application of the coating to a surface. Usually a coating (e.g., a paint) will comprise a colorizing agent (e.g., a pigment), which usually functions to alter an optical property of a coating and/or film. A coating will often comprise an additive, which is a composition incorporated into a coating to (a) reduce and/or prevent the development of a physical, chemical, and/or aesthetic defect in the coating and/or film; (b) confer some additional desired property to a coating and/or film; or (c) a combination thereof. Examples of an additive include an accelerator, an adhesion promoter, an antifloating agent, an antiflooding agent, an antifoaming agent, an antioxidant, an anti-skinning agent, a buffer, a catalyst, a coalescing agent, a corrosion inhibitor, a defoamer, a dehydrator, a dispersant, a drier, an electrical additive, an emulsifier, a film-formation promoter, a fire retardant, a flow control agent, a gloss aid, a leveling agent, a light stabilizer, a marproofing agent, a matting agent, a neutralizing agent, a preservative, a rheology modifier, a slip agent, a viscosity control agent, a wetting agent, or a combination thereof. The content for an individual coating additive in a coating generally is 0.0001% to 20.0%, including all intermediate ranges and combinations thereof. However, in many instances it is preferred if the concentration of a single additive in a coating comprises between 0.0001% and 10.0%, including all intermediate ranges and combinations thereof.

Some of the usual types of components of paints and coatings are summarized as follows: Binders (oil-based (e.g., oils, alkyd resins, oleoresinous binders, and fatty acid epoxy esters; polyester resins; modified cellulose; polyamide; amidoamine; amino resins; urethanes; phenolic resins; epoxy resins; polyhydroxyether; acrylic resins; polyvinyl binders; rubber resins; bituminous; polysulfide and silicone); Liquid Components (solvents; thinners; diluents; plasticizers; and water (e.g., hydrocarbons; oxygenated solvents; chlorinated hydrocarbons, nitrated hydrocarbons, other organic liquids)); Colorants (pigments and dyes); Additives (preservatives (e.g., biocides/bactericides/fungicides/algaecides); wetting agents; buffers (e.g., ammonium bicarbonate, both monobasic and dibasicphosphate buffers, Trizma base and zwitterionic buffers); rheology modifiers; defoamers; catalysts (e.g., driers, acids, bases, urethane catalysts)); anti-skinning agents; light stabilizers; corrosion inhibitors; dehydrators; electrical additives; and anti-insect additives. Preservatives serve to reduce or prevent the deterioration of a coating and/or film by a microorganism, by acting as a biocide, which kills an organism, a biostatic, which reduces or prevents the growth of an organism, or a combination of effects. Examples of a biocide include, for example, a bactericide, a fungicide, an algaecide, or a combination thereof.

Additional utilities for liquid coatings comprising certain synthetic peptides, including but not limited to antifungal peptides such as, but not limited to, EntV and those peptides of SEQ ID NO: 3-10), include but are not limited to increase inhibition of in-can mold growth; prevent deterioration of a latex liquid paint or coating; coating a surface to inhibit fungus infestation and growth; a method of treating a fungus-infested surface; method of impregnating a porous substrate to inhibit fungus growth and coating a fruit or grain storage vessel to inhibit mold; adhesives, sealants and elastomers containing antifungal peptides; antifungal textile finish; polymer-linked antifungal peptides; and a kit for preparing an antifungal coating.

### D. MEDICAL USE OF ANTIFUNGAL PEPTIDES

In some embodiments, the compositions provided are especially useful where conditions are conducive to fungal disease development and where control of fungal growth is preferably accomplished with compositions which are not toxic to non-fungal cells Management of disseminated fungal infections continues to present major clinical challenges. The limited spectrum of available antifungal agents contributes to both the development of acquired resistance and the rise in incidence of previously rare but intrinsically resistant pathogens. Consequently, there is an unacceptably high mortality rate in patients with fungal infections. Antifungal discovery is complicated by the similar cell biology of fungi and mammals; as a result, there are only three classes of drugs for systemic infections, the newest of which was discovered nearly 40 years ago and approved for clinical use in 2001. This illustrates the challenges of developing new antifungals, despite the dire need for them.

Life-threatening fungal infections are seen nearly exclusively in immunodeficient patients, with healthy individuals routinely exposed to these pathogens. Candida albicans is archetypal: it is a ubiquitous, mammalian commensal of the gastrointestinal and urogenital tracts as well as the skin and is rarely isolated from environmental sources (Hallen-Adams and Suhr, 2017). The normally benign association of most C. albicans with the human host raises the prospect that successful antifungal therapy need not involve eradication, but rather a restoration of microbial balance, so as to confine it to a non-pathogenic association. Consequently, significant effort has been expended to understand the normal and pathological interactions of C. albicans with the host. Less attention has been paid to how these interactions may be impacted by other components of the microbiome, but C. albicans has been observed to have both synergistic and antagonistic relationships with various bacterial species, including human pathogens such as Staphylococcus aureus, Pseudomonas aeruginosa, Streptococcus mutans and Enterococcus faecalis. It stands to reason that C. albicans, often considered to be an obligate mammalian commensal, would have evolved specific responses to its bacterial neighbors in the microbiome and is thus a platform for uncovering molecular signals that may have therapeutic potential.

The inventors had previously identified that the Gram-positive bacterium E. faecalis antagonizes the virulence of C. albicans. The effect on C. albicans is in part mediated by EntV, a bacteriocin produced by E. faecalis as a pre-pro-peptide. Processing includes removal of a signal peptide, formation of a disulfide bond, and cleavage by an extracellular protease.

There are several significant obstacles to further development of EntV as an antifungal agent. Chemical synthesis of a 68 amino acid protein that contains a required disulfide bond is nontrivial and attempts to produce this recombinantly in bacteria have failed due to toxicity. This is perhaps due to its bacteriocin activity, which poses the potential for off-target effects on the bacterial microbiome. Thus, the inventors sought to further characterize the structural determinants of the antifungal activity of EntV to separate the bacteriocin and antifungal functions in hopes of identifying smaller antifungal peptides that could be generated more efficiently and thus more cost effectively

To this end the inventors characterized the three-dimensional structure of the secreted form of EntV, prior to extracellular cleavage. Functional analysis of one alpha helix with significant antifungal, but no bacteriocin activity has been identified. Further work identified and characterized shorter fragments of the helix, down to 12 amino acids, that exhibited full antifungal activity at nanomolar concentrations in both in vitro assays and animal models of candidiasis and have identified a 10-mer variant that retains function. Thus, provided herein are anti-fungal peptides useful for the treatment or prevention of fungal infections. After obtaining EntV structural data, the inventors determined that antifungal activity was isolated to a single α-helix that can be reduced to 10-12 amino acids with full to partial activity in multiple in vitro, ex vivo, and in animal virulence models. Based on these findings the inventors further identified and characterized additional peptides with antifungal properties.

The structure revealed that EntV consists of seven alpha helices, six of which form a “clasping palm” structure that encloses the seventh helix. Functional analysis of the constituents of this structure identified this seventh helix, α7, as having significant antifungal activity. It was also determined that shorter fragments of the helix, down to 12 amino acids (SEQ ID NO:4), could exhibit full antifungal activity at nanomolar concentrations in most assays, with a 10-mer variant (SEQ ID NO: 6) retained some function. The 12 amino acid peptide (SEQ ID NO:4), was sufficient to inhibit adhesion to surfaces and biofilm formation in vitro and in a rat catheter coating model in vivo. The wild-type 12-mer peptide (SEQ ID NO: 4) also protected C. elegans from C. albicans infection and mice from oropharyngeal candidiasis (OPC). Incubation of C. albicans with the peptide prior to intravenous injection provided substantial protection against disseminated candidiasis. The wild-type 12-mer peptide (SEQ ID NO:4) is thus an anti-fungal agent for the treatment or prevention of fungal infections.

Several of these peptides can prevent infection and some were effective in an oral candidiasis model to treat infection. Several of these peptides reduced fungal biofilm adhesion in vitro, as well as, in implanted intravenous catheters. Finally, some of these peptides were effective in a systematic model that mimics deadly human bloodstream infections.

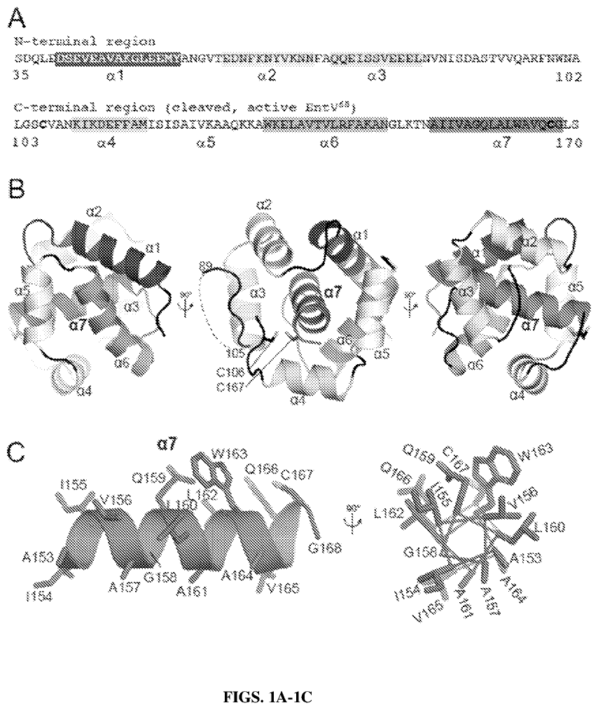

Structure of unprocessed EntV reveals “clasping palms” enclosing a C-terminal α7 helix. EntV was previously characterized as a secreted protein that undergoes multiple processing events. The 170 amino acid translated protein is directed to the secretion system by a signal sequence in its N-terminus. The signal peptide is cleaved during secretion releasing a 136 amino acid protein (FIG. 1A) (Brown et al., 2019; Dundar et al., 2015; Swe et al., 2007). The protein is further cleaved in half by the extracellular protease GelE, generating the active form of EntV which comprises the 68 amino acid C-terminus (Brown et al., 2019; Dundar et al., 2015; Graham et al., 2017). A disulfide bond formed between two cysteines in the C-terminus (C106 and C167 in FIG. 1A) was also shown to be necessary for activity (Brown et al., 2019).

TABLE 1

| | | | |
|---|---|---|---|
| | | Median | |
| | | survival | |
| Trial # | Treatment | (days) | Statistics |

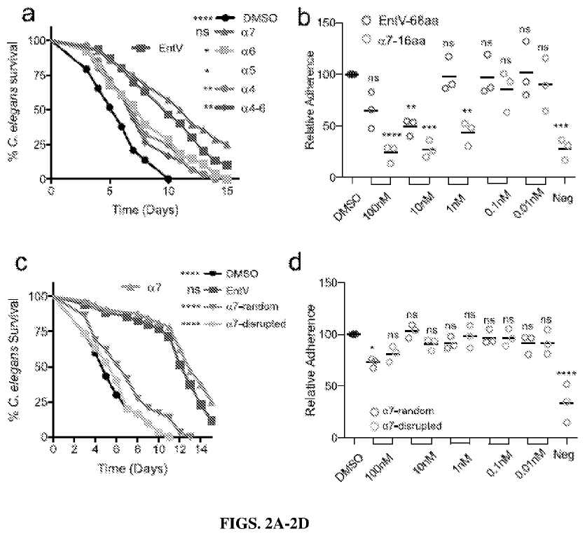

Data for FIG. 2A: C. albicans infected C. elegans treated
with various c-terminal EntV fragments

| 1 | EntV | 11 | |
|---|---|---|---|
| 2 | EntV | 9 | |
| 3 | EntV | 11 | |
| | | | p-value vs. EntV |
| 1 | DMSO | 7 | p < 0.0001 |
| 2 | DMSO | 6 | p < 0.0001 |
| 3 | DMSO | 6 | p < 0.0001 |
| 1 | α7 | 11 | p = 0.4134 |
| 2 | α7 | 10 | p = 0.6002 |
| 3 | α7 | 12 | p = 0.4292 |
| 1 | α6 | 9 | p = 0.0833 |
| 2 | α6 | 7 | p = 0.0566 |
| 3 | α6 | 8 | p = 0.0421 |
| 1 | α5 | 8 | p = 0.0161 |
| 2 | α5 | 6 | p < 0.0001 |
| 3 | α5 | 9 | p = 0.0443 |
| 1 | α4 | 11 | p = 0.7212 |
| 2 | α4 | 9 | p = 0.1302 |
| 3 | α4 | 10 | p = 0.1294 |
| 1 | α4-6 | 8 | p = 0.0036 |
| 2 | α4-6 | 6.5 | p = 0.0004 |
| 3 | α4-6 | 9 | p = 0.0025 |

Data for FIG. 2C: C. albicans infected C. elegans treated
with random or disrupted α7
Data for FIG. 2C: C. albicans infected C. elegans treated
with random or disrupted α7

| 1 | α7 | 13 | |
|---|---|---|---|
| 2 | α7 | 14 | |
| | | | p-value vs. α7 |
| 1 | DMSO | 5 | p < 0.0001 |
| 2 | DMSO | 5 | p < 0.0001 |
| 1 | EntV | 13 | p = 0.1666 |
| 2 | EntV | 12 | p = 0.2202 |
| 1 | α7 - random | 7 | p < 0.0001 |
| 2 | α7 - random | 7 | p < 0.0001 |
| 1 | α7 - disrupt | 6 | p < 0.0001 |
| 2 | α7 - disrupt | 6 | p < 0.0001 |

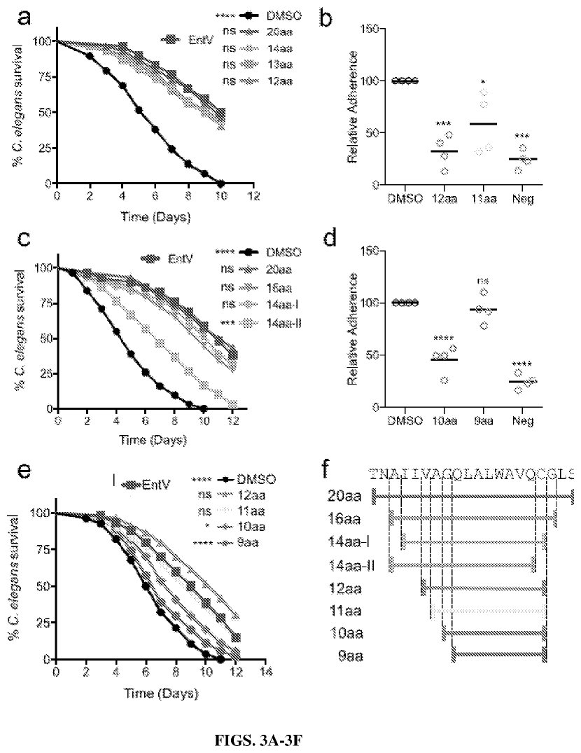

Data for FIG. 3A: C. albicans infected C. elegans treated
with various truncations of α7 retained protectivity

| 1 | EntV | 12 | |
|---|---|---|---|
| 2 | EntV | 11 | |
| 3 | EntV | 10 | |
| | | | p-value vs. EntV |
| 1 | DMSO | 6 | p < 0.0001 |
| 2 | DMSO | 6.5 | p < 0.0001 |
| 3 | DMSO | 6 | p < 0.0001 |
| 1 | 20aa | 12 | p = 0.4307 |
| 2 | 20aa | 11 | p = 0.9221 |
| 3 | 20aa | 10 | p = 0.7721 |
| 1 | 14aa | 11 | p = 0.8162 |
| 2 | 14aa | 11 | p = 0.7172 |
| 3 | 14aa | 9.5 | p = 0.4591 |
| 1 | 13aa | 10.5 | p = 0.4295 |
| 2 | 13aa | 10 | p = 0.3286 |
| 3 | 13aa | 10 | p = 0.5702 |
| 1 | 12aa | 10.5 | p = 0.4314 |
| 2 | 12aa | 10 | p = 0.1733 |
| 3 | 12aa | 9.5 | p = 0.3797 |

Data for FIG. 3C: C. albicans infected C. elegans treated
with 14aa-II truncation of α7 lost protectivity

| | | | |
|---|---|---|---|
| 1 | EntV | 12 | |
| 2 | EntV | 11 | |
| 3 | EntV | 11 | |
| 1 | DMSO | 6 | p < 0.0001 |
| 2 | DMSO | 5 | p < 0.0001 |
| 3 | DMSO | 6 | p < 0.0001 |
| 1 | 20aa | 12 | p = 0.4307 |
| 2 | 20aa | 11.5 | p = 0.7958 |
| 3 | 20aa | 11.5 | p = 0.6215 |
| 1 | 16aa | 10 | p = 0.0048 |
| 2 | 16aa | 10 | p = 0.2951 |
| 3 | 16aa | 9 | p = 0.0696 |
| 1 | 14aa-I | 12 | p = 0.8162 |
| 2 | 14aa-I | 11 | p = 0.5478 |
| 3 | 14aa-I | 11 | p = 0.6679 |
| 1 | 14aa-II | 7 | p < 0.0001 |
| 2 | 14aa-II | 7 | p < 0.0001 |
| 3 | 14aa-II | 9 | p < 0.0001 |

Data for FIG. 3E: C. albicans infected C. elegans treated
with 9aa truncation of α7 lost protectivity

| | | | |
|---|---|---|---|
| 1 | EntV | 9.5 | |
| 2 | EntV | 10 | |
| 3 | EntV | 10 | |
| 1 | DMSO | 6.5 | p < 0.0001 |
| 2 | DMSO | 7 | p < 0.0001 |
| 3 | DMSO | 7 | p < 0.0001 |
| 1 | 12aa | 11 | p = 0.0489 |
| 2 | 12aa | 10 | p = 0.6065 |
| 3 | 12aa | 10 | p = 0.6066 |
| 1 | 11aa | 9 | p = 0.2991 |
| 2 | 11aa | 10 | p = 0.9968 |
| 3 | 11aa | 10 | p = 0.9968 |
| 1 | 10aa | 8 | p = 0.0127 |
| 2 | 10aa | 9 | p = 0.2217 |
| 3 | 10aa | 9 | p = 0.2922 |
| 1 | 9aa | 7 | p < 0.0001 |
| 2 | 9aa | 7.5 | p = 0.0008 |
| 3 | 9aa | 7.5 | p = 0.0008 |

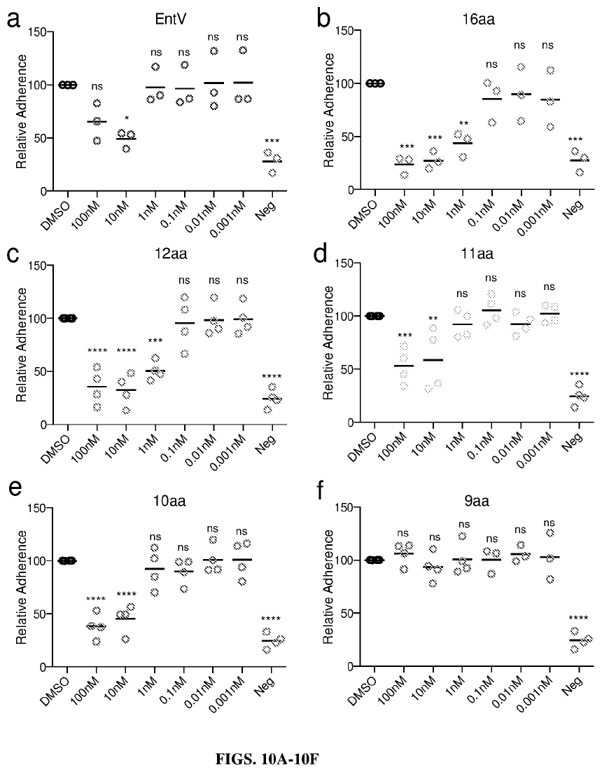

Data for FIG. 9A: C. albicans infected C. elegans treated
with α7-EE was less effective

| | | | |
|---|---|---|---|
| 1 | α7 | 10.5 | |
| 2 | α7 | 12 | |
| 1 | DMSO | 6 | p < 0.0001 |
| 2 | DMSO | 6 | p < 0.0001 |
| 1 | EntV | 10.5 | p = 0.9407 |
| 2 | EntV | 11 | p = 0.7091 |
| 1 | α7-EE | 7 | p = 0.0003 |
| 2 | α7-EE | 7.5 | p = 0.0040 |
| 1 | α7-II | 11 | p = 0.8210 |
| 2 | α7-II | 12 | p = 0.6767 |

Data for FIG. 9C: C. albicans infected C. elegans treated
with 12aa C->S and 12aa C-ALK

| 1 | 12aa | 12 | |
|---|---|---|---|
| 2 | 12aa | 11 | |
| | | | p-value vs. 12aa |
| 1 | DMSO | 7 | p < 0.0001 |
| 2 | DMSO | 6 | p < 0.0001 |
| 1 | C->S | 7 | p < 0.0001 |
| 2 | C->S | 7 | p < 0.0001 |
| 1 | ALK | 12 | p = 0.3551 |
| 2 | ALK | 9 | p = 0.1030 |

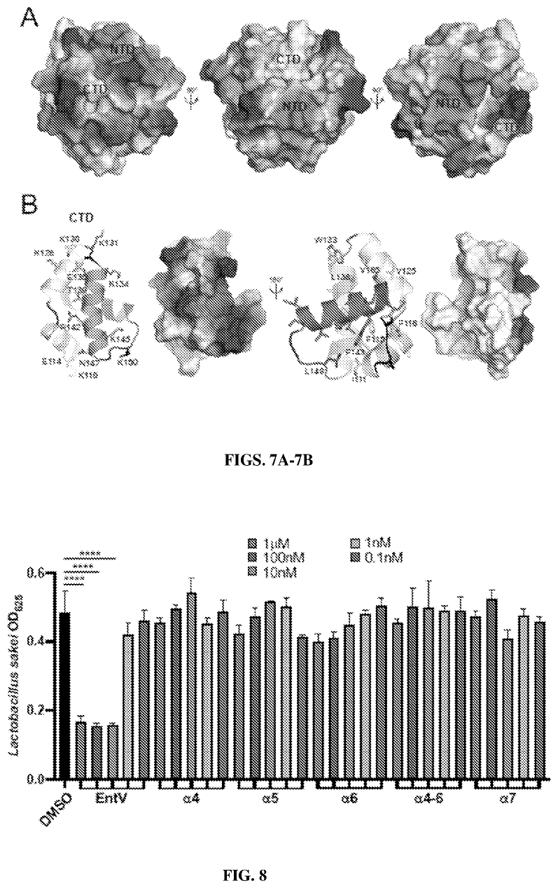

To characterize the basis for the antimicrobial properties of EntV, its structure was visualized by X-ray crystallography. Unprocessed EntV¹³⁶ was recombinantly purified from an E. coli strain as a selenomethionine-derivatized protein and crystallized. The structure was determined using single anomalous dispersion (SAD) phasing to 1.8 Å resolution, allowing for high resolution analysis of the structure. All residues were visible in the electron density except for the first two amino acids (35-36), the last two (169-170) and an internal region corresponding to residues 90-104; this latter region notably corresponds to the GelE cleavage site, and it was postulated that there is natural flexibility in this region. The predicted disulfide bond between C106 and C167 was not observed in the structure due to the necessity of producing the protein under reducing conditions in the E. coli cytosol. However, the two cysteine residues are co-localized within 3.4 Å, consistent with the ability to form an intramolecular disulfide bond. Interestingly, the structure resembled two “clasping palms” comprised of three a-helices each (FIG. 1B). The first three helices are in the N-terminus of EntV whereas the second three are part of the C-terminus. The two palms enclose a final C-terminal helix, α7 (FIGS. 1A and 1B). A strong charge asymmetry between the external faces of the two palms (FIG. 7A) was noted. The NTD (N-terminal domain) external face is negatively charged, while the CTD (C-terminal domain) external face is positively charged. The positive charge of the external face of the CTD is due to the presence of many lysine and arginine residues, while in contrast, the interior face that interacts with α7 is hydrophobic/neutral (FIG. 7B). α7 itself is strongly hydrophobic, as 11 of its 16 residues are hydrophobic sidechains (FIG. 1C). These observations are consistent with the sheltering of the hydrophobic α7 in the interior of the EntV¹³⁶ structure.

TABLE 2
X-ray crystallographic statistics.

| | |
|---|---|
| PDB code | 7ROA |

Data collection

| | |
|---|---|
| Space group | C2 |

Unit cell

| a, b, c (Å) | 62.39, 32.06, 49.99 | |
|---|---|---|
| α, β, γ, (°) | 90, 90.86, 90 | |
| Resolution, Å | 50.00-1.80 | |
| R_{merge}^{a} | 0.138 | (0.671)* |
| Rₚᵢₘ | 0.055 | (0.290) |
| CC_{1/2} | 0.913 | (0.618) |
| I/σ(I) | 14.2 | (6.9) |
| Completeness, % | 98.6 | (99.1) |
| Redundancy | 6.2 | (5.2) |

Refinement

| Resolution, Å | 31.2-1.82 | |
|---|---|---|
| No. unique reflections: | 8823, 685 | |
| working, test | | |
| R-factor/free R-factor^{b} | 17.7/17.5 | (20.5/26.9) |

No. refined atoms

| | |
|---|---|
| Protein | 904 |
| Water | 87 |

B-factors

| | |
|---|---|
| Protein | 24.6 |
| Water | 39.5 |

r.m.s.d.

| | |
|---|---|
| Bond lengths, Å | 0.006 |
| Bond angles, ° | 0.858 |

*values in brackets refer to highest resolution shells.
^{a}R_{merge} = ΣₕₖₗΣⱼ|I_{hkl.j} − [symbol] Iₕₖₗ [symbol] |/ΣₕₖₗΣⱼI_{hk, j}, where I_{hkl, j} and [symbol] Iₕₖₗ [symbol] are the jth and mean measurement of the intensity of reflection j.
^{b}Rₚᵢₘ = Σₕₖₗ√(n/n − 1) Σⁿⱼ₌₁|I_{hkl.j} − [symbol] Iₕₖₗ [symbol] |/ΣₕₖₗΣⱼI_{hk, j}
^{c}value refers to highest resolution shell.
^{d}R = Σ|Fₚ^{obs} − Fₚ^{calc}|/ΣFₚ^{obs}, where Fₚ^{obs} and Fₚ^{calc} are the observed and calculated structure factor amplitudes, respectively.

The α7 helix is sufficient to protect C. elegans and inhibit surface adhesion. Based on this structural characterization, fragments of EntV were examined to identify those structural moieties that retained antifungal activity. Synthetic peptides were generated of each of the four alpha helices (plus two flanking amino acids) from the active C-terminus of EntV—α4, α5, α6 and α7 (FIG. 1A) as well as an α4-α6 construct. The peptides were tested for their ability to protect C. elegans against C. albicans infection as established in previous work (Cruz et al., 2013; Graham et al., 2017). As shown in FIG. 2A, the α7 fragment at a concentration of 1 nM protected C. elegans from C. albicans infection as well or better than full-length, active EntV⁶⁸ (FIG. 2A and Table 1). The other peptide fragments also showed modest protection.

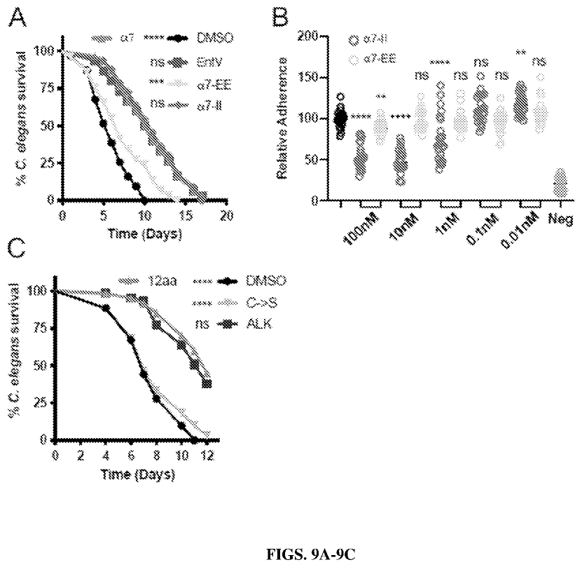

As EntV was originally characterized for its antibacterial activity against some Gram-positive species, the peptides were additionally tested for their ability to inhibit the growth of Lactobacillus sakei in liquid culture. As shown in FIG. 8, EntV abrogated the growth of L. sakei at concentrations as low as 10 nM. However, none of the EntV fragments had any activity in this assay at any of the concentrations tested. These data indicated that the antibacterial and antifungal effects of EntV are separable.

Having previously demonstrated that the full-length EntV reduced fungal burden and invasion in a mouse model of oropharyngeal candidiasis (OPC). This, like many manifestations of Candida infections, requires adhesion of the fungal cell to surfaces, both biotic and abiotic, whereupon biofilm structures are commonly formed. A model in which binding to plastic surfaces is a measure of the initial stage of biofilm differentiation. In this assay, cells are incubated in polystyrene 96 well plates with the peptides in PBS for one hour and then overlaid with artificial saliva media for 90 minutes before washing and quantitation of adhesion using crystal violet. Full-length EntV significantly reduced adhesion to polystyrene (FIG. 2B). The core 16aa α7 helix had further increased efficacy, with significant activity remaining at 1 nM (FIG. 2B). The magnitude of the reduction was equivalent that of the negative control (‘neg’ in FIG. 2B), a notably non-adherent and non-hyphal mutant lacking the CPH1 and EFG1 transcriptional regulators (Lo et al., 1997).

Variants of the α7 peptide were designed to probe the elements that were required for activity. To identify the specific sequence and/or the helical structure enabling activity, the inventors randomized the sequence of α7 in two ways. First, using the peptide folding prediction program PEP-FOLD 3 (Lamiable et al., 2016; Shen et al., 2014; Thévenet et al., 2012), the inventors generated peptides in which the sequence was randomized in a manner predicted to either retain or disrupt the helical structure. Neither peptide provided any significant protection in the worm assay (FIG. 2C), suggesting sequence specificity to the observed activity. Consistent with this, the scrambled peptides also largely lost the ability to inhibit adhesion (FIG. 2D). Indicating that antifungal activity results from the specific sequence and not a generic helical motif.

It was observed that while most of the residues that comprise the 16 amino acid core of α7 are hydrophobic, the three polar residues (two glutamines and one cysteine) are on the same face of the helix as shown in a helical wheel projection (FIG. 1C). This cysteine forms a disulfide bond that is required for the activity of the 68 amino acid peptide (Brown et al., 2019; Swe et al., 2007). Hypothesizing that the polar nature of the glutamines might be important and synthesized peptides in which these were both changed either to non-polar isoleucine or to charged glutamic acid. Surprisingly, the isoleucine substitution had no effect on activity in the worm or adhesion assays. In contrast, the glutamic acid variant was notably less effective (FIGS. 9A-B). Thus, the antifungal activity does not require the helix to be amphipathic.

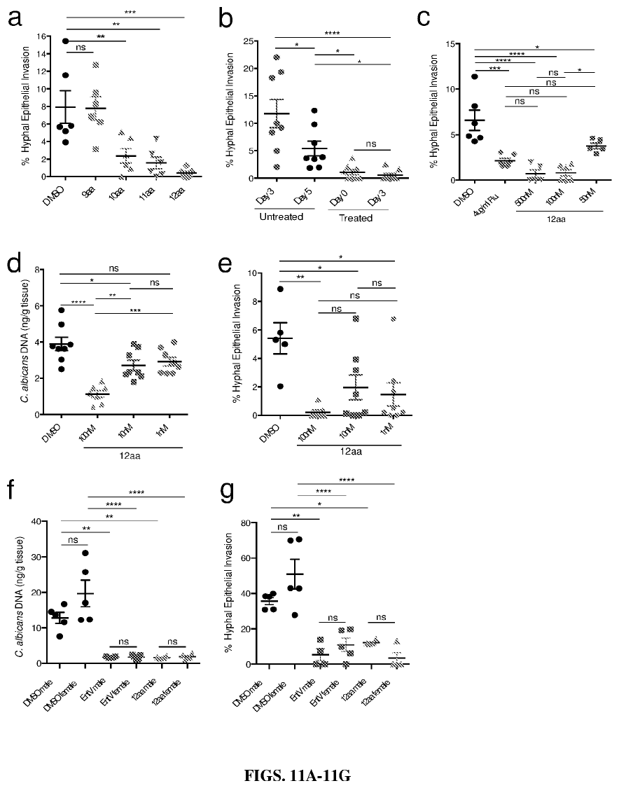

Shorter peptides retain activity. To further understand the features of α7 necessary for its activity, the inventors sought to identify the minimal length that still retained antifungal properties. Peptides were iteratively generated of decreasing length and found that peptides down to 12 amino acids retained full activity in the nematode and adhesion assays (FIGS. 3A-B, Table 1, FIG. 10). Two 14 amino acid peptides were tested, one of which contained the cysteine residue (14aa-I; FIG. 3C) and one that did not (14aa-II). Removing the cysteine greatly reduced protection in the worm model. Additionally, mutating the cysteine to a serine in the 12aa peptide abrogated activity (FIG. 9C and Table 1). However, alkylating the cysteine had no effect, suggesting that the reactivity of the cysteine sulfhydryl is not required. Further reducing the peptide size to 11 or 10 amino acids attenuated activity somewhat in both assays, while a 9-mer peptide was inactive (FIG. 3B, D, E, Table 1, FIG. 10). The tested peptides are illustrated in FIG. 3F. These data suggest that a 12-mer of the α7 helix, retaining a full three turns of the α-helix and ending with cysteine-167, is as effective in protecting against C. albicans infection in the worm model and inhibiting surface adhesion as full-length EntV.

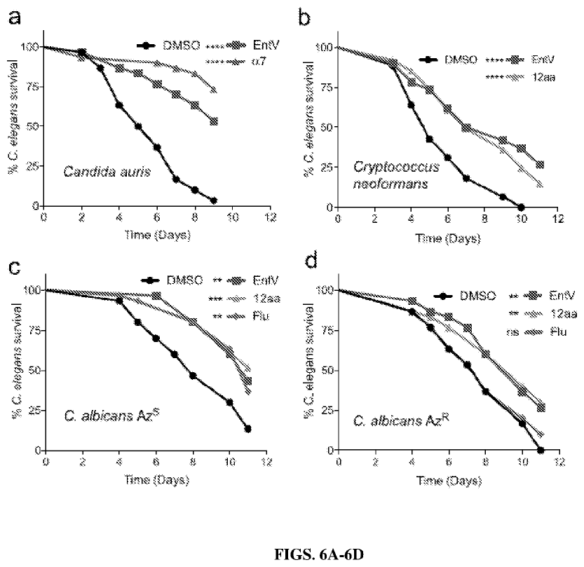

EntV peptides have activity against other fungal species and strains. Full length EntV68 can inhibit biofilm formation by other Candida species including C. tropicalis, C. parapsiolosis and C. glabrata. Based on these observations, whether EntV and derivative peptides protected C. elegans from other fungal species and drug resistant strains of C. albicans was tested. Candida auris has been defined by the CDC as an emerging pathogen of concern, causing infection in immunocompromised patients that are hard to treat due to high-levels of intrinsic drug-resistance. As shown in FIG. 6A, treatment of a C. auris strain with α7 protected animals from death. A leading cause of death in HIV/AIDS patients due to fungal meningitis is C. neoformans and the 12aa version of the α7 fragment protected C. neoformans infected worms from death (FIG. 6B). C. neoformans is in the phylum basidiomycota and is thus evolutionarily very distant from Candida species (phylum ascomycota), suggesting that the target of EntV is widely conserved amongst fungi and that the described peptides may be broadly effective therapies against fungal infections. Paired clinical isolates of C. albicans, in which fluconazole-resistance had been gained following in-patient evolution were tested and it was determined that animals infected with both the original strain as well as the fluconazole-resistant descendent were protected by EntV (FIG. 6C).

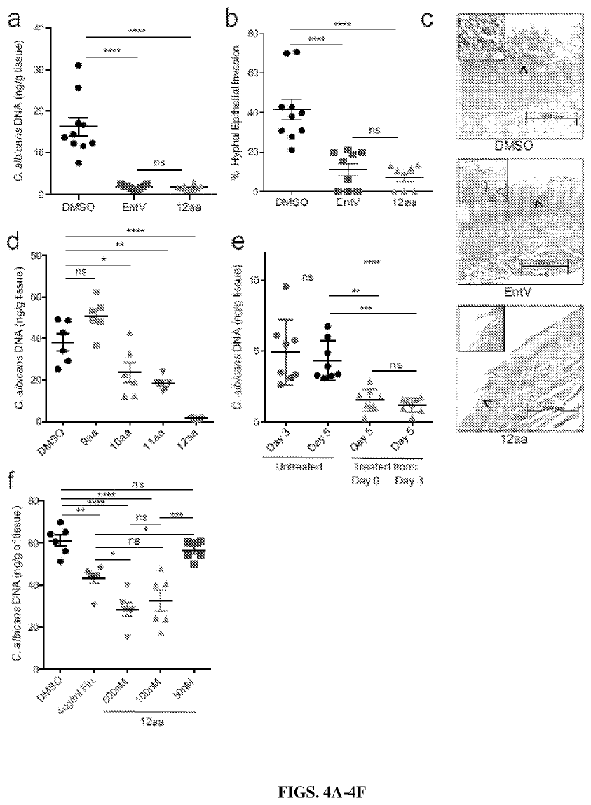

The wild-type 12-mer is protective in a mouse model of oropharyngeal candidiasis (OPC). The 12-mer (SEQ ID NO: 4) of the α7 helix was tested for efficacy in protecting against C. albicans oral infection in mice, a model in which EntV was previously shown to be effective. Briefly, corticosteroid-suppressed mice were orally inoculated with C. albicans and provided with water containing vehicle (0.01% DMSO), EntV, or the 12-mer peptide (100 nM). After five days, fungal burden was assessed in the tongue using qPCR and epithelial invasion by histological examination. As shown in FIG. 4A, treatment with EntV significantly reduced the fungal burden, as observed in the previous study (Graham et al., 2017), and treatment with the 12-mer was equally effective. Epithelial invasion of the tongues was assessed via histology and determined that the wild-type 12 amino acid peptide (SEQ ID NO: 4) was at least as effective as the full length EntV (FIGS. 4B-C). In contrast, to the extensive hyphal morphology and invasion in control animals, C. albicans is found mostly in the yeast morphological state when associated with tissue from the animals treated with EntV or the 12-mer (FIG. 4C). Overall, these data showed that the 12-mer is as effective as EntV⁶⁸ in abrogating an oropharyngeal infection of C. albicans.

The shorter peptides were examined for activity and, consistent with the C. elegans and adhesion assays, the peptides of wild-type 11 and 10 amino acids (SEQ ID NOS: 5 and 6, respectively) reduced fungal burden in the OPC model, but not to the same degree as the 12-mer. The 9mer exhibited no significant protection (FIG. 4D). Hyphal invasion was also most reduced following treatment with the 12-mer, with some reduction observed in animals exposed to the wild-type 10-mer and 11mer (SEQ ID NOS: 6 and 5, respectively). The 9mer had no effect (FIG. S5A). The results support the conclusion that the 12-mer is the optimal fragment length, comparable to or better than EntV⁶⁸ in all assays.

In these studies, as well as in the previous study, ad libitum treatment with EntV or its truncated variants commenced as soon as the animals recovered from the oral inoculation with C. albicans by adding peptide to drinking water. To determine if the 12-mer has efficacy as a treatment after the establishment of an infection, mice were inoculated, but did not administer peptide until day three (FIG. 4E). A group of untreated infected animals was also sacrificed at day three to check for successful establishment of the infection. Animals treated with the wild-type 12-mer (SEQ ID NO:4) starting at day three and sacrificed on day five had significantly less fungal burden compared to both groups of untreated animals. The level of reduction was not significantly different from those animals who had been treated from day zero (FIG. 4E).

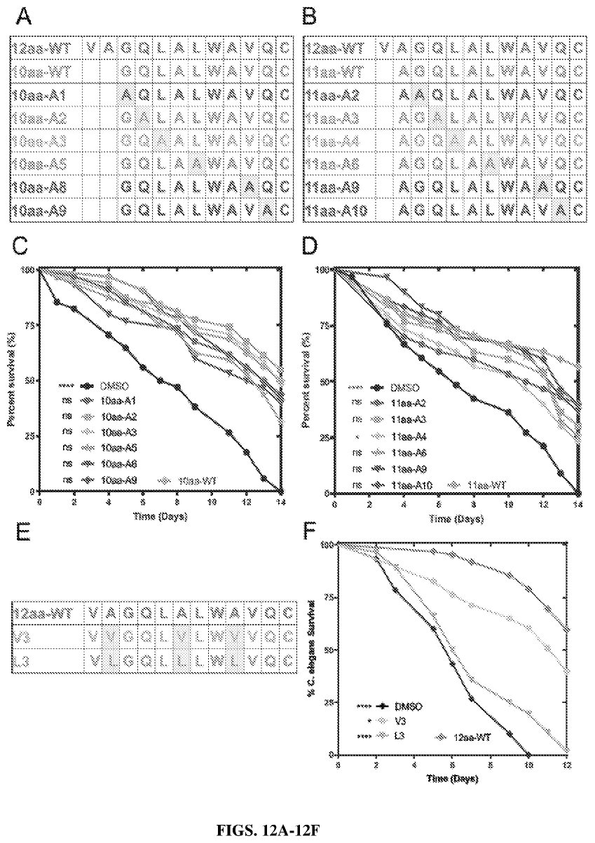

The same patterns were observed when hyphal invasion was used as the measurement of infection severity (FIG. 11B). These results indicate that the 12-mer has efficacy in treating established OPC.

A comparison of the efficacy of the wild-type 12-mer (SEQ ID NO: 4) relative to fluconazole, an antifungal agent recommended to treat OPC. At the standard dose of 100 nM, the 12-mer (SEQ ID NO: 4) was even more effective at reducing fungal burden than 4 μg/mL fluconazole, 8-times the MIC for this strain, though this was only statistically significant when the concentration of EntV was increased to 500 nM (FIG. 4F). Hyphal invasion was slightly less prevalent after treatment with 100 nM and 500 nM relative to fluconazole (FIG. 11C). A lower dose (50 nM) was also tested, which resulted in loss of efficacy in terms of fungal burden, though there was still a significant lowering of hyphal invasion (FIG. 4F and 10C). Overall, these data showed that oral treatment with 100 nM 12-mer (SEQ ID NO: 4) was comparable or better than treatment with fluconazole, a standard and accepted chemotherapeutic.

Given the sustained efficacy of the 12-mer (SEQ ID NO: 4) to reduce epithelial invasion at 50 nM, yet lower concentrations were tested. At 10 nM and 1 nM there is only a slight reduction in fungal burden on the tongue, but the reduction in invasion remains dramatic (FIG. 11D-E). The EntV 12-mer appears to promote a commensal, non-invasive, association with the host epithelium at concentrations well below those needed to see a reduction in fungal numbers. Finally, there were no differences in fungal burden, invasion, or EntV/12-mer efficacy between male and female mice (FIGS. 11F-G).

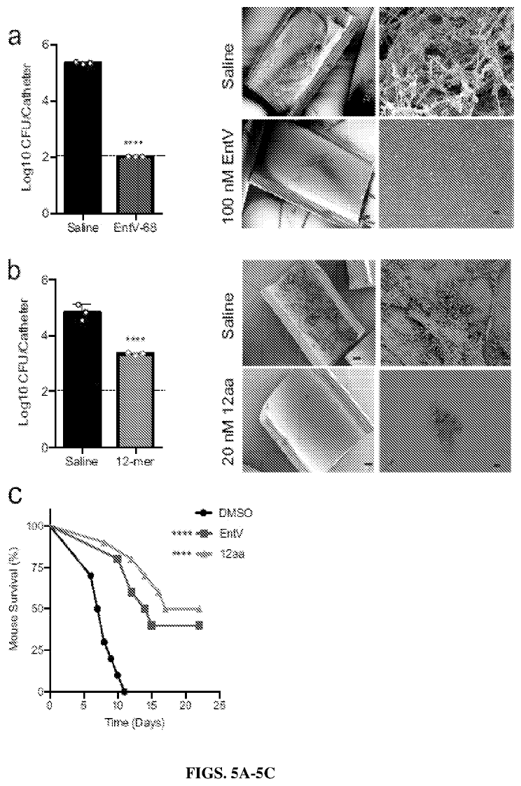

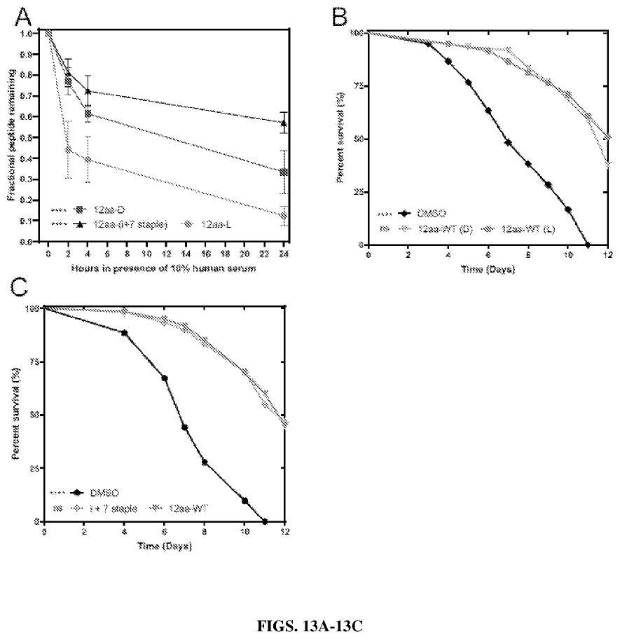

The wild-type 12-mer (SEQ ID NO: 4) is protective in a rat venous catheter model of C. albicans infection. C. albicans can form prodigious biofilms on implanted medical devices and the presence of an intravenous catheter is, by itself, a significant risk factor for developing disseminated candidiasis. These infections are notoriously difficult to treat and are associated with the drug resistance characteristic of biofilms. Thus, full-length EntV was tested in a model in which an intravenous catheter is implanted into the jugular vein of rats and inoculated with C. albicans with or without the peptide. After 24 hours, the catheter is removed and sectioned to assess fungal burden by plating for CFUs and by scanning electron microscopy (SEM). Catheters treated with 100 nM EntV were essentially sterile, with fungal burden below the limit of detection (FIG. 5A). As the minimal peptide length was determined, this model utilized along with the 12-mer peptide, this time at a lower concentration to probe the extent of protection. Even as low as 20 nM, the 12-mer (SEQ ID NO: 4) reduced fungal burdens by 1.5 logs (FIG. 5B) and SEMs showed the catheter surfaces to be largely clear of fungal biofilm (FIG. 12F). The data indicate that the EntV peptides are very effective in preventing C. albicans biofilm formation on abiotic surfaces and has potential as a treatment and/or a preventative of C. albicans medical device-associated infections.

The wild-type 12-mer is protective in a systemic mouse model of C. albicans infection. Systemic bloodstream infections by C. albicans are the most life-threatening to patients. Mortality rates from disseminated candidiasis exceed 40% and new treatments are in dire need. Since EntV impairs hyphal morphogenesis and biofilm formation rather than killing C. albicans (Graham et al., 2017), it was unclear if EntV and EntV derivatives would be protective in a systemic infection model. To increase the chances of observing an effect, C. albicans were pre-incubated with 100 nM of EntV or the 12-mer for two hours prior to intravenous (IV) injection of 1×10⁵ CFU. CFUs were measured both before and after the two-hour incubation and there was no significant difference, indicating that the EntV versions were not toxic to C. albicans. The median survival of animals injected with C. albicans alone was 7.5 days and all animals had expired by 11 days. In striking contrast, animals injected with C. albicans that had been pre-incubated with EntV or the 12-mer (SEQ ID NO: 4) survived much longer and about half the animals were still alive when the experiment was ended in accordance with the protocol at day 22 (FIG. 5C).

Structural studies demonstrated that the pro-peptide of EntV (EntV136) forms a clasping palm motif that cages the α7 helix in a manner that portends its importance. Indeed, informed by this data, the antifungal activity was isolated to this single α-helix and just 10-12 amino acids have full to partial activity in multiple in vitro, ex vivo, and animal virulence models. Excitingly, these peptides not only prevented the onset of infection but, in the oral candidiasis model, effectively treated an established infection. They also reduced adhesion in vitro and in intravenous catheters implanted into rats, which models a key route of infection for human patients. Finally, EntV and the 12-mer displayed efficacy in a systematic mouse model that mimics deadly human bloodstream infections.

### E. ADDITIONAL ANTIFUNGAL PEPTIDES

The inventors then generated a combinatorial library based on the sequence of the wild-type 10-mer (SEQ ID NO:6), which had lower activity than the 12-mer, (SEQ ID NO:4) and performed unbiased screens of this library to identify gain-of-function 10-mers with antifungal activity equal to or greater than the 12-mer (see Table 3).

A series of mutated 10aa and the 11aa variants were generated based on 12 aa variant of the EntV peptide described above. The ability of these peptides to protect C. elegans from C. albicans infection were determined and compared to the wild type sequences for the 10aa and 11aa variants. All had some activity, but none were clearly superior.

Alanine scan and mutational analysis of EntV truncated peptides. The peptides generated for both the 10aa and the 11aa variants are shown in FIGS. 1A and 1B. Residues that were not mutated included the native alanine residues found in the wild-type sequence and the tryptophan found in the middle of the sequence, which is critical for determining peptide concentration. The ability of these peptides to protect C. elegans from C. albicans infection is shown in comparison to the wild type sequences for the 10aa and 11aa variants, respectively, in FIGS. 12C and 12D.

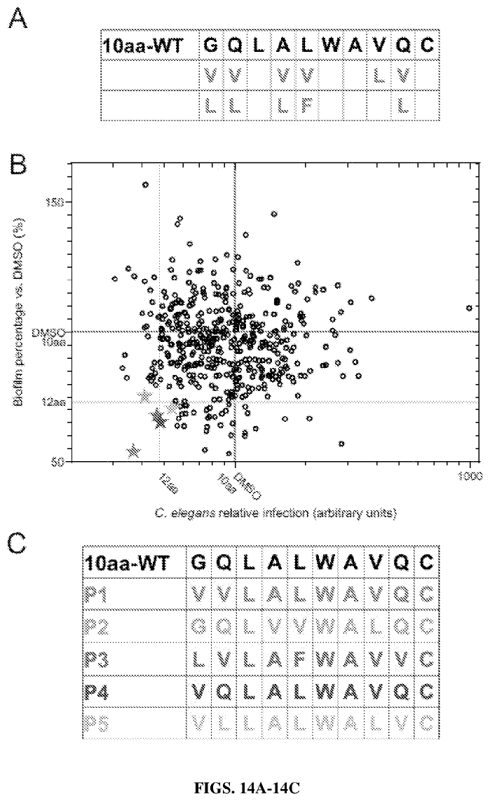

Peptide modifications to improve stability. The 12aa variant was modified to see if stability could be further improved. The first modification was generating the peptide with D-form amino acids, which are inherently more resistant to proteases and peptidases due to the nature of the peptide bonds. The D-form peptide had notably improved peptide stability compared to the naturally occurring L-form peptide, with approximately 35% remaining after 24 hours in the presence of human serum (FIG. 13A). A variant of the 12aa peptide that uses a hydrocarbon staple was generated. The purpose of this staple is two-fold, it can lock the conformation of the secondary structure for the helical peptide, and it also sterically hinders accessibility of the peptide backbone to proteases and peptidases. This modification exploits the spacing of alanine residues in the sequence to introduce a hydrocarbon staple by synthesizing the peptide with modified alanine residues. These types of hydrocarbon staples can only be used in short, helical peptides and must occur in the canonical i+3, i+4, or i+7 positions to coincide with the number of residues in one or two turns of the helix. As shown in FIG. 13A, this modification was significantly stabilizing, resulting in approximately 60% of the original peptide still being intact after the 24-hour incubation. Importantly, neither modification caused loss of antifungal activity. Both the 12aa D-form peptide and the stapled peptide remained as effective as the original in protecting C. elegans against infection with C. albicans as evidenced in FIG. 13B and 13C.

High-throughput screening of a combinatorial peptide library. In an attempt to identify a variant of shorter sequence length but which retained activity the 10aa variant was chosen as the parent sequence to generate a combinatorial peptide library as a first step in the process of synthetic molecular evolution. The method of synthetic molecular evolution takes peptides of known sequence and uses them as a parent sequence to create a combinatorial library of new peptides that are then screened for enhanced activity based on the screening parameters. Several positions along the native 10aa sequence were chosen as flexible positions to incorporate more hydrophobic residues; split-and-recombine chemistry was carried out using solid-phase peptide synthesis to generate a library of 486 novel peptides.

High-throughput screening of this library was performed by employing established assays to assess antifungal agent efficacy. The first was a C. elegans infection assay and the live/dead stain Sytox Orange to assess the status of the nematodes post-infection. The second assay was a C. albicans biofilm reduction assay. By using these assays in concert, each individual peptide was assessed using two approaches that probe different aspects of C. albicans virulence.

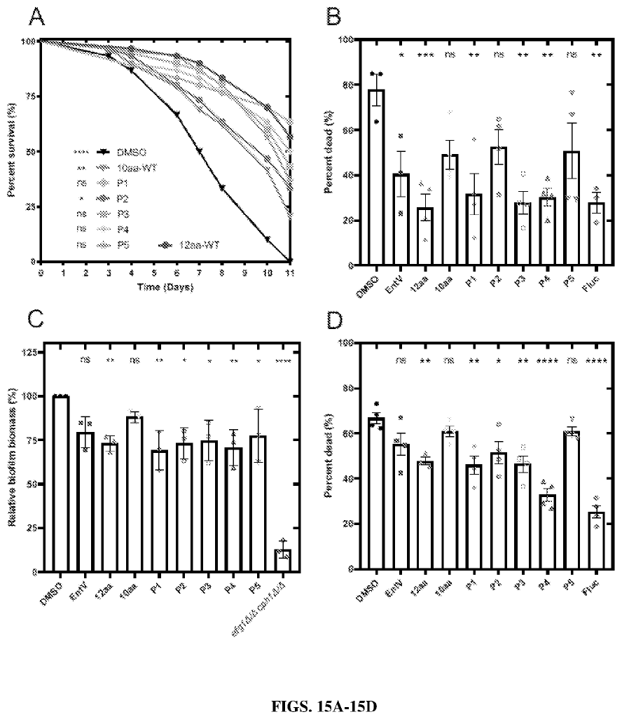

Most of the peptides from the library were no more active than the 10aa parent peptide, however, 5 peptides displayed enhanced activity comparable to the 12aa peptide. The five peptides were sequenced by Edman degradation and their divergence from the parent sequence is presented in Table 3 and FIG. 14C.

TABLE 3
Short peptides with antifungal activity

| | | |
|---|---|---|
| EntV Alpha 7 | TNAIIVAGQLALWAVQCG | SEQ ID NO: 3 |
| helix sequence | | |
| wt-12-mer | VAGQLALWAVQC | SEQ ID NO: 4 |
| wt-11 mer | AGQLALWAVQC | SEQ ID NO: 5 |
| wt-10-mer | GQLALWAVQC | SEQ ID NO: 6 |
| P1 10-mer | VVLALWAVQC | SEQ ID NO: 7 |
| P2 10-mer | GQLVVWALQC | SEQ ID NO: 8 |
| P3 10-mer | LVLAFWAVVC | SEQ ID NO: 9 |
| P4 10-mer | VQLALWAVQC | SEQ ID NO: 10 |
| P5 10-mer | VLLALWALVC | SEQ ID NO: 11 |
| P6 10-mer | VQLALWALVC | SEQ ID NO: 12 |

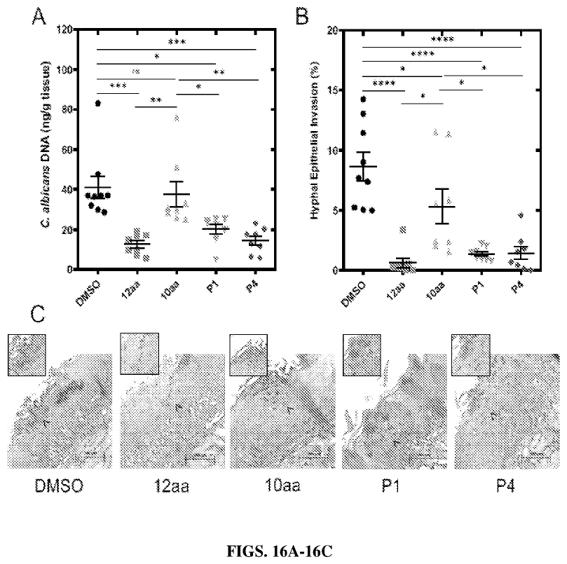

Four of the five 10 aa library hits (P1, P3, P4, and P5) were as effective in protecting the animals as the original 12aa peptide; the differences in survival were not statistically significant (FIG. 15A). Compared to the untreated DMSO control, it was found that three of the mutants P1, P3, and P4 showed statistically significant antifungal activity in comparison to the DMSO control and in contrast with the 10aa parent sequence (FIG. 15B, 18).

The new batches of peptides were then tested for their ability to reduce biofilm biomass. Compared to the untreated DMSO control and in contrast to the 10aa parent variant, all five of the mutant peptides demonstrated statistically significant antifungal activity. (FIG. 15C). The mutant peptides showed approximately a 20-30% decrease in biofilm biomass. The experiment included the efg1Δ/Δ cph1Δ/Δ strain of SC5314 C. albicans that is unable to form biofilms as a negative control. These results demonstrate that the mutant 10aa peptides have activity better than the original 10aa variant and that their activity resembles that of the 12aa variant.

Finally, the mutant peptides were tested against a different fungal species, Cryptococcus neoformans. While the 10aa parent sequence was not effective, P1 through P4 all had significant antifungal activity when compared to the untreated DMSO control (FIG. 15D, 19). In conclusion, the mutant peptides identified from the library demonstrated broad-spectrum antifungal activity comparable to the 12aa parent.

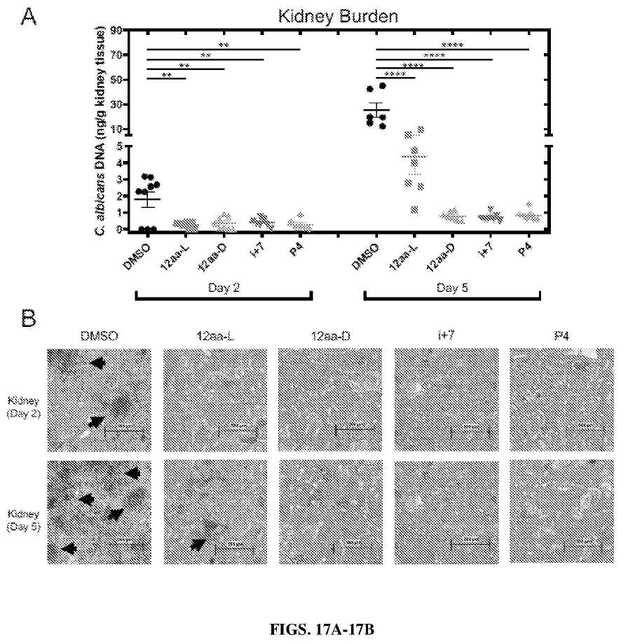

Peptides P1 and P4 were tested in an established model of tissue-invasive oropharyngeal candidiasis, which was used to validate the efficacy of several EntV peptides in previous studies. A biofilm-based infection was established by placing an inoculation swab of C. albicans into the mouth of the mice sublingually for 75 minutes, then mice from each group were given water containing the peptides that was consumed ad libitum for five days. After five days, the mice were sacrificed, and the tongue tissue was processed for qPCR and histological analyses. As shown in FIGS. 16A and 16B both the fungal burden and the % hyphal invasion were significantly reduced by treatment with the P1 and P4 peptides as compared to the DMSO control and to the 10aa parent peptide (FIGS. 16A and 16B).

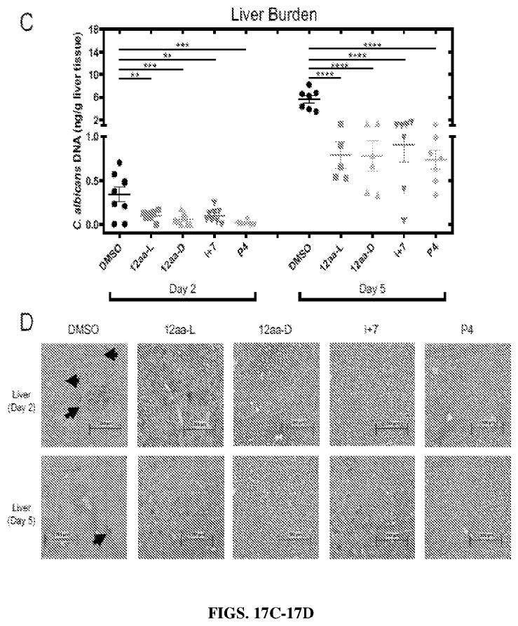

Antifungal efficacy of two mutant peptides in a murine model of systemic candidiasis. Next, select peptides were tested in a murine systemic model of candidiasis that resembles human bloodstream infections with C. albicans. Among the new 10aa variants identified in the screen, P4 was chosen because it was more effective than P1 in reducing fungal burden in the OPC model (FIGS. 16A). Also tested were the peptides that were more stable in human serum, 12aa-D and 12aa (I+7 staples) (FIG. 13) to test if their increased stability improved efficacy. A bloodstream Candidiasis infection was established by inoculating outbred ICR mice with a tail vein injection containing 5×10⁵ CFUs of C. albicans pre-incubated with or without peptide for 2 hours prior to the injection. Endpoints of 2 days and 5 days post-infection were chosen to assess C. albicans burden in the kidney and liver of the mice. The kidney and liver were chosen because bloodstream C. albicans infections demonstrate tropism to those organs for colonization. At the two endpoints, the mice were sacrificed, and the right kidney and medial lobe of the liver were extracted from each mouse. The tissue was then cut in half to be processed for qPCR and for histological analyses. As shown in FIG. 17A, both on Day 2 and on Day 5, the Candida burden was reduced in the kidneys with statistical significance for all peptide-preincubated inoculations compared to the DMSO (untreated) control. In addition to fungal burden, histological images were collected and used to determine representative examples of hyphal formation in the kidneys are presented in FIG. 17B. Similarly, when assessing the liver Candida burden, a statistically significant reduction was observed for all peptide-preincubated inoculations compared to the DMSO control (FIG. 17C). As done with the kidneys, and histological images were collected and representative examples of hyphal formation in the liver were obtained. Clusters of C. albicans with a high density of hyphal formation were observed in the DMSO control, while little C. albicans is observed in the treated samples.

Therapeutic Formulation and Administration for Medical Indications: Therapeutics containing pharmaceutical compositions comprising the antifungal peptides described and/or nucleotide sequences that encode the amino acid sequences of the antifungal peptides described herein nucleotide sequences that encode the amino acid sequences of the antifungal peptides, can be administered to a patient at therapeutically effective doses of pharmaceutical preparations used to treat or ameliorate conditions fungal infections. In further aspects a therapeutically effective dose refers to that amount of the composition sufficient to result in any amelioration or retardation of fungal disease symptoms or progression in a mammal.

Toxicity and therapeutic efficacy of such antifungal peptide compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

Compositions which exhibit large therapeutic indices are preferred. While compositions that exhibit toxic side effects may be used, care should be taken to design a delivery system that targets such compounds to the site of affected tissue in order to minimize potential damage to uninfected cells and, thereby, reduce side effects.

Data obtained from cell culture assays and animal studies can be used in formulating a range of dosages for use in humans. The dosage of such compositions is preferably within a range of circulating concentrations that include the ED₅₀ with little or no toxicity. The dosage may vary within this range depending upon the dosage form employed and the route of administration utilized. For any compositions used in the methods of the embodiments, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

When the therapeutic treatment of disease is contemplated, the appropriate dosage can also be determined using animal studies to determine the maximal tolerable dose, or MTD, of a bioactive agent per kilogram weight of the test subject. In general, at least one animal species tested is mammalian. Those skilled in the art regularly extrapolate doses for efficacy and avoiding toxicity to other species, including humans. Before human studies of efficacy are undertaken, Phase I clinical studies in normal subjects help establish safe doses.

Additionally, the bioactive agent (such as the antifungal peptides described herein) may be complexed with a variety of well-established compounds or structures that, for instance, enhance the stability of the bioactive agent, or otherwise enhance its pharmacological properties (e.g., increase in vivo half-life, reduce toxicity, etc.).

Pharmaceutical compositions for use in accordance with the present embodiments can be formulated in conventional manner using one or more physiologically acceptable carriers or excipients.

The above therapeutic compositions will be administered by any number of methods known to those of ordinary skill in the art including, but not limited to, administration by inhalation; by subcutaneous (sub-q), intravenous (I.V.), intraperitoneal (I.P.), intramuscular (I.M.), or intrathecal injection; or as a topically applied agent (transderm, ointments, creams, salves, eye drops, and the like). Thus, the compositions can be formulated for administration by inhalation or insufflation (either through the mouth or the nose) or oral, buccal, parenteral or rectal administration.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). Tablets can be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration can be suitably formulated to give controlled release of the active composition. For buccal administration the compositions can take the form of tablets or lozenges formulated in conventional manner. For administration by inhalation, the compositions for use according to the embodiments are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compositions can be formulated for parenteral administration by injection, e.g., by bolus injection or continuous infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compositions can also be formulated for rectal administration such as suppositories or retention enemas, e.g., containing conventional suppository bases such as cocoa butter or other glycerides.

The compositions can also be formulated for vaginal administration such as suppositories or creams, e.g., containing conventional suppository bases such as cocoa butter or other glycerides or cream bases such as mineral oil combined with emulsion stabilizers or white soft paraffin.

The compositions can be formulated for topical application to susceptible organisms such as topical creams and ointments in the case of animals, or as sprays, fogs, mists, powders and the like in the case of plants.

In addition to the formulations described previously, the compounds may also be formulated as a depot preparation. Such long-acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt. The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

In some embodiments, various methods of microsphere preparation may be used depending upon the hydrophilic or hydrophobic nature of the peptide composition to be encapsulated. Wang, H.T., et al. 1991, “Influence of formulation methods on the in vitro controlled release of protein from poly (ester) microspheres,” J. of Controlled Release 17:23-25.

### Stabilizing Peptides

In some embodiments, a variety of modifications can be made to the peptides as long as antifungal activity is retained. Some modifications may be used to increase the intrinsic antifungal activity of the peptide. Other modifications may facilitate handling of the peptide. Peptide functional groups that may typically be modified include hydroxyl, amino, guanidinium, carboxyl, amide, phenol, imidazol rings or sulfhydryl. Typical reactions of these groups include but are not limited to acetylation of hydroxyl groups by alkyl halides. Carboxyl groups may be esterified, amidated or reduced to alcohols. Carbodiimides or other catalysts may be used to catalyze the amidation of carboxyl groups. The amide groups of asparagine or glutamine may be deamidated under acidic or basic conditions. Acylation, alkylation, arylation or amidation reactions readily occur with amino groups such as the primary amino group of the peptide or the amino group of lysine residues. The phenolic group of tyrosine can be halogenated or nitrated. Examples where solubility of a peptide could be decreased include acylating charged lysine residues or acetylating the carboxyl groups of aspartic and glutamic acids. Peptides may be conjugated to soluble or insoluble carrier molecules to modify their solubility properties as needed and to increase the local concentrations of peptides in their target areas. Examples of soluble carrier molecules include polymers of polyethyleneglycol and polyvinylpyrrolidone. Examples of insoluble polymers include sand or other silicates or polystyrene, cellulose, or the like. Peptides may also be micro-encapsulated to enhance their stability during seed, soil, or plant application. Typically, polyester microspheres are used to encapsulate and stabilize the peptides.

In some embodiments, to enhance the activity and stability of the described antifungal peptides, they may be produced using D-amino acids or unusual amino acids instead of the natural L-residues, cyclization, glycosylation, deamination, complete removal of the first residue, N-acylation or N-formylation at the N-terminus, amidation of the C-terminus, etc. D-amino acids can increase the stability of certain of these antifungal peptides, being insensitive to common biological degradation pathways that degrade L-amino acid peptides. For instance, L-amino acid peptides may be stabilized by addition of D-amino acids at one or both of the peptide termini. D-residues may also enforce a different conformation of the peptide, and strongly influence receptor affinity and selectivity. However, biochemical pathways are available which will degrade even D-amino acids in these peptides so that long term environmental persistence is enhanced. However, where the compositions of the disclosure act rapidly or need not otherwise be stabilized, L-amino acids or mixtures of L-and D-amino acids may be useful. Furthermore, the antifungal peptides described herein may function most efficiently or be more stable as a particular stereoisomer, or as a mixed stereoisomeric compositions. N-alkylation (generally N-methylation) may enhance biologic activity of peptides from different sources by protecting from proteolysis. Peptides modified by the use of N-methyl amino acids have resulted in analogues with improved stability and pharmacological properties. Cyclic analogues of such antifungal peptides may be much more stable with respect to the native peptide by stabilizing peptide structure. Connections that may be utilized to restrain antifungal peptide structure into a cyclic framework, include but are not limited to disulfide bridges or mimics macrolactons, ether bridges or biaryl bridges, etc.

In some embodiments, a variety of modifications can be made to the peptides as long as antifungal activity is retained. Some modifications may be used to increase the intrinsic antifungal activity of the peptide. Other modifications may facilitate handling of the peptide. Peptide functional groups that may typically be modified include hydroxyl, amino, guanidinium, carboxyl, amide, phenol, imidazol rings or sulfhydryl. Typical reactions of these groups include but are not limited to acetylation of hydroxyl groups by alkyl halides. Carboxyl groups may be esterified, amidated or reduced to alcohols. Carbodiimides or other catalysts may be used to catalyze the amidation of carboxyl groups. The amide groups of asparagine or glutamine may be deamidated under acidic or basic conditions. Acylation, alkylation, arylation or amidation reactions readily occur with amino groups such as the primary amino group of the peptide or the amino group of lysine residues. The phenolic group of tyrosine can be halogenated or nitrated. Examples where solubility of a peptide could be decreased include acylating charged lysine residues or acetylating the carboxyl groups of aspartic and glutamic acids. Peptides may be conjugated to soluble or insoluble carrier molecules to modify their solubility properties as needed and to increase the local concentrations of peptides in their target areas. Examples of soluble carrier molecules include polymers of polyethyleneglycol and polyvinylpyrrolidone. Examples of insoluble polymers include sand or other silicates or polystyrene, cellulose, or the like. Peptides may also be micro-encapsulated to enhance their stability during seed, soil, or plant application. Typically, polyester microspheres are used to encapsulate and stabilize the peptides.

With insights derived from EntV structural data, antifungal activity was isolated to a single alpha helix that can be reduced to 10-12 amino acids with full to partial activity in multiple in vitro, ex vivo, and animal virulence models. These peptides not only prevented the onset of infection but, in the oral candidiasis model, effectively treated an established infection. Application of these peptides reduced adhesion in vitro and in intravenous catheters implanted into rats, which models a key route of infection for human patients. The 12-mer displayed efficacy in a systematic mouse model that mimics deadly human bloodstream infections.

No apparent toxicity was seen with the peptides in the mouse model, and the previous study observed no toxicity when EntV was incubated with human and mouse cells). These shorter peptides are no longer bacteriocins, having separated the antifungal and antibacterial activities. Furthermore, despite its robust protection against infection, EntV is not toxic to C. albicans. Growth of C. albicans was unaffected when EntV was added to shaking cultures in concentrations as high as 30 μM

### F. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Material and Methods

Strains and media. C. albicans was routinely propagated in YPD medium (1% yeast extract, 2% peptone, 2% dextrose). Most experiments used wild-type strain SC5314 (Gillum et al., 1984). For the adhesion assays the non-adherent strain HLC54 (cph1Δ::hisG/cph1Δ::hisG efg1Δ::hisG/efg1Δ::hisG-URA3-hisG ura3/ura3) (Lo et al., 1997) was used as a negative control.

Protein expression and purification. Strain CEGE1, a BL21 (DE3) derivative containing pET28a encoding EntV¹³⁶ (Graham et al., 2017), was used for overexpression. 10 mL overnight culture was diluted into 1 L M9 SeMET (selenomethionine) media containing selection antibiotic ampicillin and grown at 37° C. with shaking till OD₆₀₀ reached 1.2. The cell culture was induced with IPTG after switching temperature to 20° C. and left shaking overnight. Cell pellets were collected by centrifugation, resuspended in binding buffer [100 mM HEPES pH 7.5, 300 mM NaCl, 5 mM imidazole and 5% glycerol (v/v)] and sonicated. Insoluble debris was removed by centrifugation at 17000 rpm. Ni-NTA affinity chromatography was used for protein purification. The supernatant was loaded on a column with 4 mL Ni-NTA resin (QIAGEN), pre-equilibrated with binding buffer with 0.5 mM TCEP, washed with 120 mL washing buffer [100 mM HEPES pH 7.5, 300 mM NaCl, 30 mM imidazole, 5% glycerol (v/v) and 0.5 mM TCEP], and protein was eluted with elution buffer [100 mM HEPES pH 7.5, 300 mM NaCl, 250 mM imidazole, 5% glycerol (v/v) and 0.5 mM TCEP]. The His-tag was cleaved by adding human thrombin (5 units per mg of recombinant protein) and 2.5 mM Calcium Chloride, in overnight dialysis against buffer, containing 20 mM Tris-HCl pH 8.0, 0.1M NaCl, 0.5 mM TCEP and 5 mM Imidazol. After second Ni-NTA column the tag-free protein was dialyzed against crystallization buffer (10 mM HEPES pH 7.5, 300 mM NaCl), and the purity of the protein was analyzed by SDS-polyacrylamide gel electrophoresis.

Crystallization and Structure Determination. The EntV¹³⁸ crystal was grown at room temperature using the vapor diffusion sitting drop method using 2 μL of a 25 mg/mL protein solution, 0.1 M Tris pH 8 and 28% (w/v) PEG4K. The crystal was cryoprotected with paratone-N oil prior to flash freezing in liquid nitrogen. Diffraction data at 100K at beamline 19-ID of the Structural Biology Center of the Advanced Photon Source, Argonne National Laboratory. HKL3000 (Minor et al., 2006) was used to process two Se-Met diffraction data sets. Computational corrections for absorption in a crystal and Lorentz factor were applied (Borek et al., 2003; Otwinowski et al., 2003). Anisotropic diffraction correction was necessary for structure solution (Borek et al., 2010, 2013; Otwinowski et al., 2003). Indexing, integration, and scaling indicated C2 symmetry. The best Se-Met crystal, used in the refinement, diffracted to a nominal resolution of ˜1.8 Å but diffraction was highly anisotropic, with diffraction pattern extending to ˜1.9 Å resolution in the a direction (<I>/<σ(I)>˜2) and to a resolution much higher than 1.8 Å in the b and c directions (<I>/<σ(I)>˜19 and ˜13, respectively). Initial phases were obtained from a single Se-Met crystal in a single-wavelength anomalous diffraction (SAD) experiment by performing heavy atom search to resolution 2.2 Å using SHELXD (3). The search identified 2 Se positions with correlations coefficients: CC_{All}=31.3%, CC_{Weak}=25.9%, and C_{FOM}=57.3% with relative occupancies of 1.000 and 0.660. The handedness of the best solution was determined with SHELXE. The heavy atom positions were refined to 1.8 Å with MLPHARE (Sheldrick, 2008), with the final FOM reaching 0.185 for all observations. The density modification was performed with DM (Cowtan and Main, 1998, 1996; Cowtan and Zhang, 1999). The resulting electron density map was used as the entry model for the model building with BUCCANEER (Cowtan, 2006) and refinement with REFMAC (Murshudov et al., 1997, 1999), run within HKL3000. The resulting main chain was ˜82% complete (116 aa) with ˜79% of side chains docked into electron density maps (112 aa), and R factor=23.3% and R_{free}=24.3%. The resulting assembly was used to perform isomorphous replacement with another data set using MOLREP ((Vagin and Teplyakov, 2010) run within HKL3000, and rebuilt and refined again with BUCCANEER and REFMAC run within HKL3000. The resulting main chain was ˜90% complete (127 aa) with ˜80% of side chains docked into electron density maps (113 aa), and R factor=23.4% and Rfree=27.7%. Refinement was completed using Phenix.refine (Adams et al., 2010) and Coot (Emsley et al., 2010). B-factors were refined as anisotropic for protein atoms and isotropic for non-protein atoms and TLS parameterization was included in the refinement. Average B-factor and bond angle/length RMSD values were calculated using Phenix. All geometry was verified using the Phenix, Coot and wwPDB validation tools. The X-ray crystallographic statistics are in Table 2 and the structure was deposited to the Protein Data Bank under the accession code 7ROA.

Peptide synthesis and alkylation. Peptide synthesis was performed using standard Solid Phase Peptide Synthesis (SPPS) protocols. Starting from the C-terminal residue, the peptides were synthesized on Tentagel S-Ram 0.2-0.8 meq/g beads (from Chem Impex). FMOC protected peptide monomers (from Advanced ChemTech) were dissolved in dimethylformamide (DMF) at a 4× molar excess relative to the manufacturer's stated loading capacity. The reactions were catalyzed by the addition of Hexafluorophosphate Benzotriazole Tetramethyl Uronium (HBTU), Hydroxybenzotriazole (HOBt), and N,N-Diisopropylethylamine (DIPEA). Reaction completion for each residue was confirmed by conducting a ninhydrin test on a small sample of beads taken from the reaction vessel after washing the with DMF three times. Following completion of amino acid addition, the beads underwent a final deprotection to remove the FMOC group and then were washed with dichloromethane (DCM). Acid cleavage from the beads was accomplished by using Reagent B (88% v/v trifluoroacetic acid, 5% v/v phenol, 5% v/v ddH2O, 2% v/v triisopropylsilane). Synthesis quality was verified by performing HPLC and mass spectrometry. Prior to analysis, the peptides were ether precipitated, dissolved in DMSO, and lyophilized. Peptide alkylation was conducted by reacting a 5× molar excess of iodoacetamide to the wild-type 12-mer variant of EntV for 48 hours. The reaction was confirmed by monitoring the retention times of the peptides via HPLC. The product was then pooled by HPLC fraction collection and then dried down for use once the product weight was confirmed by mass spectrometry.

Killing of C. elegans by C. albicans. The methodology used for the C. elegans infection assays was performed, with a few modifications, as described ((Breger et al., 2007; Cruz et al., 2013; Graham et al., 2017; Peleg et al., 2008)). Briefly, C. elegans glp-4(bn2); sek-1(km-4) (Breger et al., 2007) nematodes were propagated and maintained on E. coli strain OP50(Brenner, 1974) that was seeded onto nematode growth medium (NGM) agar using standard techniques (Hope, 1999). To synchronize the animals, L1 stage worms on non-starved plates were washed off, filtered through a 10 μm filter (pluriSelect, pluriStrainer 10 μm), harvested by centrifugation, transferred to OP50 seeded plates, and grown to the L4 stage. To prepare the infection plates, C. albicans strain SC1534 (Gillum et al., 1984) was grown in YPD broth for 24 h at 37° C. with agitation. 500 μl of the culture was plated onto BHI solid medium containing gentamycin (10 μg/ml) and grown for 24 h at 37° C. The synchronized, L4 C. elegans were washed off the OP50 plates in 2 ml sterile M9 buffer and washed once, centrifuging at 750×g for 30 seconds to collect animals. Animals were infected by placing them on the SC5314 lawn for 4 h at 25° C. Following this exposure, they were washed off the plate and washed four times with 2 ml of sterile M9. The nematodes were then pipetted (˜30 per well with 2 wells per condition for a total of ˜60 worms assayed) into six-well plates with 2 ml of liquid medium (20% BHI broth and 80% M9) containing the indicated concentrations of test compounds. Plates were incubated at 25° C., and worm death was scored daily. Kaplan-Meier survival curves were generated and analyzed as described in the quantification and statistical analysis section.

C. albicans adhesion assays. The C. albicans adhesion assays were performed using similar techniques as described previously (Gulati et al., 2018). C. albicans strains were grown in YPD broth for 18 h at 30° C. with agitation and then sub-cultured for 4 h in the same conditions. Cells were then collected by centrifugation, washed twice in PBS, and adjusted to a concentration of 1×10⁷ cells/ml (measured with Countess II, Life Technologies) in PBS treated with the indicated concentrations of test compounds. The cell suspension was incubated for 1 h at 30° C. with agitation. Then 100 μl of the cell suspension was added to wells of a 96 well tissue culture treated polystyrene plate (Falcon) in addition to 100 μl of artificial saliva media (Graham et al., 2017; Wong and Sissons, 2001) containing the indicated concentrations of test compounds. The plate was then incubated at 37° C. for 90 minutes.

After incubation, the media was gently removed along with any cells that failed to adhere. The adhered cell layer was then stained for 20 minutes with 40 μl of 0.08% crystal violet solution (diluted, Sigma). The crystal violet stain was removed, and the wells were then washed three times with sterile water. Adhered cells were then destained with 200 μl of 200 proof ethanol for 20 minutes, and 100 μl of the ethanol solution was transferred to a new well for analysis. The optical density at 595 nm (OD₅₉₅) was measured using a Synergy H1 plate reader (BioTek) with Gen5 version 3.08 software (BioTek).

Lactobacillus MIC Assay. For this assay, Lactobacillus sakei ATCC 15521 (Torriani et al., 1996) was used following the methodology as described with a few modifications (Chang et al., 2011). Briefly L. sakei was grown in Lactobacilli MRS broth (Difco, Detroit, MI, USA) for 18 h at 30° C. without agitation (Malheiros et al., 2015). Following adjustment to a concentration of 1×10⁶ cells/ml, cells were diluted 1:10 into fresh MRS medium containing the indicated concentrations of EntV fragments. After 24 hours of growth at 30° C. without agitation, OD₆₂₅ readings were taken using a BioTek Cytation5 plate reader. Readings of blanks (containing fresh MRS medium) were subtracted from sample wells. Three technical replicates were averaged, and the experiment was repeated three times. Results in the L. sakei MIC assay showed that the helices alone or all three together are not enough to recapitulate antibacterial activity; thus while α7 is required, it is not sufficient.

Hemolysis assay. De-identified and pooled RBCs were purchased from Rockland Immunochemicals, Inc. RBCs were prepared at 6×10⁷ cells/mL in PBS. To 25 μL of peptide diluted appropriately to result in a final concentration of 1 nM and 1 μM, 125 μL of RBC suspension was added. The plates were gently rocked for 1 h at room temperature. The cells were then removed from suspension by centrifugation at 1000×g for 5 min. From the experimental plate, 100 μL of the supernatant was carefully withdrawn from each well and added to a new 96-well plate. The absorbance of the solutions in each well was measured at 410 nm and each well was compared to a no-treatment control and a 100% lysis control (1% SDS).

Peptide serum stability assay. One mL of RPMI supplemented with 10% (v/v) of human serum (type AB) were aliquoted and temperature-equilibrated at 37° C. for 15 min before adding a concentrated stock peptide solution resulting in a final peptide concentration of 100 μM. For each time point, 100 μL of the reaction solution was removed and added to 200 μL 96% ethanol for precipitation of serum proteins. The sample was cooled (4° C.) for 15 min and then spun at 18,000 g for 2 min to pellet the precipitated serum proteins. The reaction supernatant was analyzed using HPLC with absorbance detected at 280 nm. The peptide peak was detected first without serum treatment to assess the area under the curve for the peptide of interest. At each time point the area under the curve was calculated by HPLC and then divided by the untreated peptide area under the curve to determine the percent remaining.

Mouse OPC model. The efficacy of EntV fragments were tested in the OPC model as described (Graham et al., 2017; Solis and Filler, 2012). Mice were immunosuppressed by injecting 225 mg/kg cortisone acetate subcutaneously 1 d before inoculation, and subsequently on days 1 and 3 of the infection. Group size was 6-8. To prepare the inoculum, 1 ml of C. albicans (SC5314) overnight culture grown at 30° C. in YPD broth was washed twice in PBS before resuspension in sterile Hanks' balanced salt solution (HBSS) at a concentration of 1×10⁶ cells/mL. Calcium alginate swabs were soaked in this inoculum for 5 minutes prior to inoculation. Mice were anesthetized using ketamine and xylazine and placed on pre-warmed isothermal pads and the swabs were placed sublingually for 75 min. Mice were given additional doses of ketamine (50 mg/kg), as necessary. After inoculation, mice were given drinking water with EntV fragments or DMSO (0.01%) in the drinking water ad libitum. Mice were euthanized at 5 d after inoculation, unless noted. The tongues were excised and cut in half laterally for tissue histology and assessment of fungal burden.

For tissue histology the tongue sample was placed in 10% zinc-buffered formalin overnight and stored in 80% ethanol, before embedding in paraffin. For each tongue, 5 μm sections were prepared using a Leica microtome and stained using Periodic Acid-Schiff (PAS) stain. Epithelial invasion was measured from 40× images taken of the entire tissue section of each tongue half with the total area of epithelium and infected epithelium were measured using ImageJ (version 1.6). Measurements were totaled and expressed as a percentage of total infected epithelium relative to the entire epithelial area (Graham et al., 2017; Moyes et al., 2016).

For examination of fungal burden, qPCR was used by amplifying a 269-bp fragment of the internal transcribed spacer 2 (ITS2) between the 5.8S and 28S ribosomal RNA genes of C. albicans (Graham et al., 2017; Khot et al., 2009). DNA was extracted from homogenized tongue tissue using the Yeast DNA Extraction Kit (Thermo Scientific) according to the protocol with minor modifications. The ITS2 fragment was amplified by qPCR was performed with a CFX96 Real-Time System with a C1000 Touch thermal cycler (BioRad). C. albicans gDNA was quantified using FastStart Universal SYBR Green master mix with ROX (Roche). To screen for contamination and background fluorescence during the qPCR amplification, no template controls were used.

Intravenous infection model. The disseminated candidiasis model was performed as described (Miramon and Lorenz, 2016; Williams and Lorenz, 2020). C. albicans SC5314 was grown overnight in YPD at 30° C., then diluted to a very low concentration and grown in a second overnight. The late-log phase culture was collected by centrifugation, washed in PBS, and diluted to 5×10⁶ cells/ml in PBS containing vehicle (0.01% DMSO) or 100 nM EntV or the 12-mer peptide and incubated for 2 hours at room temperature. 10 female ICR mice (18-20 g) were inoculated via the lateral tail vein with 0.1 ml. Animals were monitored at least twice daily for 21 days for signs of moribundity.

Rat catheter model: The protocol for the rat catheter biofilm was conducted as previously described (Andes, et al., 2004). Briefly, indwelling central venous catheters in rats were inoculated with 1×10⁶ cells/mL C. albicans with or without 100 nM EntV or 20 nM 12-mer peptide (group size was 3). To quantitate fungal burden after 24 h, the catheter was removed, and 1 cm of the tip placed in a one milliliter of sterile 0.9% NaCl followed by vortexing. A 1:10 serial dilution of this wash fluid was plated on SDA and colonies counted after 24 h of growth at 35° C. For scanning electron microscopy (SEM), catheters were fixed overnight in 4% formaldehyde and 1% glutaraldehyde in PBS. The catheters were then washed with PBS and treated with 1% osmium tetroxide in PBS for 30 min. Serial ethanol washes and critical point drying were used to dry the segments before they were mounted, and palladium-gold coated. All images were taken using a SEM LEO 1530, with Adobe Photoshop CC (20.0.4 release) used for image compilation.

Animal protocols. All mouse experiments were conducted under protocols approved by the Animal Welfare Committees of the University of Wisconsin (catheter experiments) or the University of Texas Health Science Center at Houston (all other models).

Quantification and statistical analysis. GraphPad Prism 9.0 was used for all data analysis. For the fungal adhesion and bacterial MIC, assays, means of the experimental conditions were calculated and compared to the DMSO condition. Lines with error bars indicate the mean and the standard deviation (SD). Significance was determined using one-way ANOVA followed by Dunnett's multiple comparison test. For the OPC fungal burden and % hyphal invasion measurements, means of the experimental conditions were calculated and compared to other conditions as indicated in the individual panels. Lines with error bars indicate the mean and the standard error of the mean (SEM). Significance was determined using one-way ANOVA followed by Tukey's multiple comparison test. For the catheter experiments, the means and the standard deviations were calculated, and an unpaired, two-tailed t test was used to compare the treatments to the controls. Mantel-Cox log rank analysis was used to compare survival curves. The median survival and comparison values for all C. elegans survival experiments and their replicates can be found in Table 1. For all statistical tests, p values <0.05 were considered statistically significant and asterisks in the figure panels indicate the levels of significance as follows: *p<0.05, **p<0.01, ***p<0.001, ****p<0.0001.

Without further elaboration, it is believed that one skilled in the art can, using the description herein, utilize the present methods to its fullest extent. The embodiments described herein are to be construed as illustrative and not as constraining the remainder of the disclosure in any way whatsoever. While preferred embodiments have been shown and described, many variations and modifications thereof can be made by one skilled in the art without departing from the spirit and teachings of the presently disclosed methods. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they are consistent with the present disclosure set forth herein.

### G. REFERENCES

The following references, to the extent that they provide exemplary procedural or other details supplementary to those set forth herein, are specifically incorporated herein by reference.
Adams, P. D., Afonine, P. V, Bunkóczi, G., Chen, V. B., Davis, I. W., Echols, N., Headd, J. J., Hung, L.-W., Kapral, G. J., Grosse-Kunstleve, R. W., et al. (2010). PHENIX: a comprehensive Python-based system for macromolecular structure solution. Acta Crystallogr. D. Biol. Crystallogr. 66, 213-221.
Andes, D., Nett, J., Oschel, P., Albrecht, R., Marchillo, K., and Pitula, A. (2004). Development and characterization of an in vivo central venous catheter Candida albicans biofilm model. Infect. Immun. 72, 6023-6031.
Arastehfar, A., Gabaldón, T., Garcia-Rubio, R., Jenks, J. D., Hoenigl, M., Salzer, H. J. F., Ilkit, M., Lass-Flörl, C., and Perlin, D. S. (2020). Drug-Resistant Fungi: An Emerging Challenge Threatening Our Limited Antifungal Armamentarium. Antibiot. (Basel, Switzerland) 9.
Borek, D., Minor, W., and Otwinowski, Z. (2003). Measurement errors and their consequences in protein crystallography. Acta Crystallogr. D. Biol. Crystallogr. 59, 2031-2038.
Borek, D., Cymborowski, M., Machius, M., Minor, W., and Otwinowski, Z. (2010). Diffraction data analysis in the presence of radiation damage. Acta Crystallogr. D. Biol. Crystallogr. 66, 426-436.
Borek, D., Dauter, Z., and Otwinowski, Z. (2013). Identification of patterns in diffraction intensities affected by radiation exposure. J. Synchrotron Radiat. 20, 37-48.
Breger, J., Fuchs, B. B., Aperis, G., Moy, T. I., Ausubel, F. M., and Mylonakis, E. (2007). Antifungal chemical compounds identified using a C. elegans pathogenicity assay. PLoS Pathog 3, e18.
Brenner, S. (1974). The genetics of Caenorhabditis elegans. Genetics 77, 71-94.
Brown, A. O., Graham, C. E., Cruz, M. R., Singh, K. V, Murray, B. E., Lorenz, M. C., and Garsin, D. A. (2019). Antifungal Activity of the Enterococcus faecalis Peptide EntV Requires Protease Cleavage and Disulfide Bond Formation. MBio 10.
Brown, G. D., Denning, D. W., Gow, N. A. R., Levitz, S. M., Netea, M. G., and White, T. C. (2012). Hidden killers: human fungal infections. Sci. Transl. Med. 4, 165rv13.
Buda De Cesare, G., Cristy, S. A., Garsin, D. A., and Lorenz, M. C. (2020). Antimicrobial Peptides: a New Frontier in Antifungal Therapy. MBio 11.
Chang, Y.-C., Tsai, C.-Y., Lin, C.-F., Wang, Y.-C., Wang, I.-K., and Chung, T.-C. (2011). Characterization of tetracycline resistance lactobacilli isolated from swine intestines at western area of Taiwan. Anaerobe 17, 239-245.
Cowtan, K. (2006). The Buccaneer software for automated model building. 1. Tracing protein chains. Acta Crystallogr. D. Biol. Crystallogr. 62, 1002-1011.
Cowtan, K., and Main, P. (1998). Miscellaneous algorithms for density modification. Acta Crystallogr. D. Biol. Crystallogr. 54, 487-493.
Cowtan, K. D., and Main, P. (1996). Phase combination and cross validation in iterated density-modification calculations. Acta Crystallogr. D. Biol. Crystallogr. 52, 43-48.
Cowtan, K. D., and Zhang, K. Y. (1999). Density modification for macromolecular phase improvement. Prog. Biophys. Mol. Biol. 72, 245-270.
Cruz, M. R., Graham, C. E., Gagliano, B. C., Lorenz, M. C., and Garsin, D. A. (2013). Enterococcus faecalis inhibits hyphal morphogenesis and virulence of Candida albicans. Infect Immun 81, 189-200.
Dundar, H., Brede, D. A., La Rosa, S. L., El-Gendy, A. O., Diep, D. B., and Nes, I. F. (2015). The fsr Quorum-Sensing System and Cognate Gelatinase Orchestrate the Expression and Processing of Proprotein EF_1097 into the Mature Antimicrobial Peptide Enterocin O16. J Bacteriol 197, 2112-2121.
Emsley, P., Lohkamp, B., Scott, W. G., and Cowtan, K. (2010). Features and development of Coot. Acta Crystallogr. D. Biol. Crystallogr. 66, 486-501.
Fisher, M. C., Gurr, S. J., Cuomo, C. A., Blehert, D. S., Jin, H., Stukenbrock, E. H., Stajich, J. E., Kahmann, R., Boone, C., Denning, D. W., et al. (2020). Threats Posed by the Fungal Kingdom to Humans, Wildlife, and Agriculture. MBio 11.
Garsin, D. A., and Lorenz, M. C. (2013). Candida albicans and Enterococcus faecalis in the gut: Synergy in commensalism? Gut Microbes 4.
Gillum, A. M., Tsay, E. Y., and Kirsch, D. R. (1984). Isolation of the Candida albicans gene for orotidine-5′-phosphate decarboxylase by complementation of S. cerevisiae ura3 and E. coli pyrF mutations. Mol Gen Genet 198, 179-182.
Graham, C. E., Cruz, M. R., Garsin, D. A., and Lorenz, M. C. (2017). Enterococcus faecalis bacteriocin EntV inhibits hyphal morphogenesis, biofilm formation, and virulence of Candida albicans. Proc Natl Acad Sci U S A 114, 4507-4512.
Gulati, M., Lohse, M. B., Ennis, C. L., Gonzalez, R. E., Perry, A. M., Bapat, P., Arevalo, A. V., Rodriguez, D. L., and Nobile, C. J. (2018). In vitro Culturing and Screening of Candida albicans Biofilms. Curr. Protoc. Microbiol. 50, e60.
Hallen-Adams, H. E., and Suhr, M. J. (2017). Fungi in the healthy human gastrointestinal tract. Virulence 8, 352-358.
Hermann, C., Hermann, J., Munzel, U., and Ruchel, R. (1999). Bacterial flora accompanying Candida yeasts in clinical specimens. Mycoses 42, 619-627.
Khot, P. D., Ko, D. L., and Fredricks, D. N. (2009). Sequencing and analysis of fungal rRNA operons for development of broad-range fungal PCR assays. Appl Env. Microbiol 75, 1559-1565.
Lamiable, A., Thévenet, P., Rey, J., Vavrusa, M., Derreumaux, P., and Tufféry, P. (2016). PEP-FOLD3: faster de novo structure prediction for linear peptides in solution and in complex. Nucleic Acids Res. 44, W449-54.
Lo, H. J., Kohler, J. R., DiDomenico, B., Loebenberg, D., Cacciapuoti, A., and Fink, G. R. (1997). Nonfilamentous C. albicans mutants are avirulent. Cell 90, 939-949.
Malheiros, P. S., Sant'Anna, V., Todorov, S. D., and Franco, B. D. G. M. (2015). Optimization of growth and bacteriocin production by Lactobacillus sakei subsp. sakei2a. Brazilian J. Microbiol. [Publication Brazilian Soc. Microbiol. 46, 825-834.
Minor, W., Cymborowski, M., Otwinowski, Z., and Chruszcz, M. (2006). HKL-3000: the integration of data reduction and structure solution--from diffraction images to an initial model in minutes. Acta Crystallogr. D. Biol. Crystallogr. 62, 859-866.
Miramon, P., and Lorenz, M. C. (2016). The SPS amino acid sensor mediates nutrient acquisition and immune evasion in Candida albicans. Cell Microbiol 18, 1611-1624.
Moyes, D. L., Wilson, D., Richardson, J. P., Mogavero, S., Tang, S. X., Wernecke, J., Hofs, S., Gratacap, R. L., Robbins, J., Runglall, M., et al. (2016). Candidalysin is a fungal peptide toxin critical for mucosal infection. Nature 532, 64-68.
Murshudov, G. N., Vagin, A. A., and Dodson, E. J. (1997). Refinement of macromolecular structures by the maximum-likelihood method. Acta Crystallogr. D. Biol. Crystallogr. 53, 240-255.
Murshudov, G. N., Vagin, A. A., Lebedev, A., Wilson, K. S., and Dodson, E. J. (1999). Efficient anisotropic refinement of macromolecular structures using FFT. Acta Crystallogr. D. Biol. Crystallogr. 55, 247-255.
Nguyen, L. T., Haney, E. F., and Vogel, H. J. (2011). The expanding scope of antimicrobial peptide structures and their modes of action. Trends Biotechnol. 29, 464-472.
Nobile, C. J., and Johnson, A. D. (2015). Candida albicans Biofilms and Human Disease. Annu Rev Microbiol 69, 71-92.
Otwinowski, Z., Borek, D., Majewski, W., and Minor, W. (2003). Multiparametric scaling of diffraction intensities. Acta Crystallogr. A. 59, 228-234.
Pappas, P. G., Kauffman, C. A., Andes, D. R., Clancy, C. J., Marr, K. A., Ostrosky-Zeichner, L., Reboli, A. C., Schuster, M. G., Vazquez, J. A., Walsh, T. J., et al. (2016). Clinical Practice Guideline for the Management of Candidiasis: 2016 Update by the Infectious Diseases Society of America. Clin. Infect. Dis. an Off. Publ. Infect. Dis. Soc. Am. 62, e1-50.
Peleg, A. Y., Tampakakis, E., Fuchs, B. B., Eliopoulos, G. M., Moellering Jr., R. C., and Mylonakis, E. (2008). Prokaryote-eukaryote interactions identified by using Caenorhabditis elegans. Proc Natl Acad Sci U S A 105, 14585-14590.
Perfect, J. R. (2017). The antifungal pipeline: a reality check. Nat. Rev. Drug Discov. 16, 603-616.
Perlin, D. S., Rautemaa-Richardson, R., and Alastruey-Izquierdo, A. (2017). The global problem of antifungal resistance: prevalence, mechanisms, and management. Lancet. Infect. Dis. 17, e383-e392.
Pfaller, M. A., and Diekema, D. J. (2007). Epidemiology of invasive candidiasis: a persistent public health problem. Clin. Microbiol. Rev. 20, 133-163.
Rolling, T., Hohl, T. M., and Zhai, B. (2020). Minority report: the intestinal mycobiota in systemic infections. Curr. Opin. Microbiol. 56, 1-6.
Romo, J. A., and Kumamoto, C. A. (2020). On Commensalism of Candida. J. Fungi (Basel, Switzerland) 6.
Santus, W., Devlin, J. R., and Behnsen, J. (2021). Crossing Kingdoms: How the Mycobiota and Fungal-Bacterial Interactions Impact Host Health and Disease. Infect. Immun. 89.
Sheldrick, G. M. (2008). A short history of SHELX. Acta Crystallogr. A. 64, 112-122.
Shen, Y., Maupetit, J., Derreumaux, P., and Tuffery, P. (2014). Improved PEP-FOLD Approach for Peptide and Miniprotein Structure Prediction. J. Chem. Theory Comput. 10, 4745-4758.
Solis, N. V, and Filler, S. G. (2012). Mouse model of oropharyngeal candidiasis. Nat Protoc 7, 637-642.
Swe, P. M., Heng, N. C., Ting, Y. T., Baird, H. J., Carne, A., Tauch, A., Tagg, J. R., and Jack, R. W. (2007). ef1097 and ypkK encode enterococcin V583 and corynicin JK, members of a new family of antimicrobial proteins (bacteriocins) with modular structure from Gram-positive bacteria. Microbiology 153, 3218-3227.
Thévenet, P., Shen, Y., Maupetit, J., Guyon, F., Derreumaux, P., and Tufféry, P. (2012). PEP-FOLD: an updated de novo structure prediction server for both linear and disulfide bonded cyclic peptides. Nucleic Acids Res. 40, W288-93.
Torriani, S., Van Reenen, G. A., Klein, G., Reuter, G., Dellaglio, F., and Dicks, L. M. (1996). Lactobacillus curvatus subsp. curvatus subsp. nov. and Lactobacillus curvatus subsp. melibiosus subsp. nov. and Lactobacillus sake subsp. sake subsp. nov. and Lactobacillus sake subsp. carnosus subsp. nov., new subspecies of Lactobacillus curvatus Abo-El. Int. J. Syst. Bacteriol. 46, 1158-1163.
Vagin, A., and Teplyakov, A. (2010). Molecular replacement with MOLREP. Acta Crystallogr. D. Biol. Crystallogr. 66, 22-25.
Vale-Silva, L. A., Coste, A. T., Ischer, F., Parker, J. E., Kelly, S. L., Pinto, E., and Sanglard, D. (2012). Azole resistance by loss of function of the sterol Δ5,6-desaturase gene (ERG3) in Candida albicans does not necessarily decrease virulence. Antimicrob. Agents Chemother. 56, 1960-1968.
Vasicek, E. M., Berkow, E. L., Flowers, S. A., Barker, K. S., and Rogers, P. D. (2014). UPC2 is universally essential for azole antifungal resistance in Candida albicans. Eukaryot. Cell 13, 933-946.
Williams, R. B., and Lorenz, M. C. (2020). Multiple Alternative Carbon Pathways Combine To Promote Candida albicans Stress Resistance, Immune Interactions, and Virulence. MBio 11.
Wisplinghoff, H., Ebbers, J., Geurtz, L., Stefanik, D., Major, Y., Edmond, M. B., Wenzel, R. P., and Seifert, H. (2014). Nosocomial bloodstream infections due to Candida spp. in the USA: species distribution, clinical features and antifungal susceptibilities. Int. J. Antimicrob. Agents 43, 78-81.
Wong, L., and Sissons, C. (2001). A comparison of human dental plaque microcosm biofilms grown in an undefined medium and a chemically defined artificial saliva. Arch Oral Biol 46, 477-486.
Zeise, K. D., Woods, R. J., and Huffnagle, G. B. (2021). Interplay between Candida albicans and Lactic Acid Bacteria in the Gastrointestinal Tract: Impact on Colonization Resistance, Microbial Carriage, Opportunistic Infection, and Host Immunity. Clin. Microbiol. Rev. 34, e0032320.

What is claimed is:

1. A peptide or polypeptide consisting or consisting essentially of an anti-fungal portion of the α7 domain of EntV, wherein the peptide or polypeptide comprises an amino acid sequence of SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, or SEQ ID NO: 12.

2. The peptide or polypeptide of claim 1, wherein the peptide or polypeptide comprises an amino acid sequence of SEQ ID NO: 4.

3. The peptide or polypeptide of claim 1, wherein the peptide or polypeptide comprises or consists of an amino acid sequence of SEQ ID NO: 7 or SEQ ID NO: 10.

4. The peptide or polypeptide of claim 1, wherein the peptide or polypeptide is a fusion protein of SEQ ID NOS: 3-12 with a heterologous peptide or peptide sequence.

5. The peptide or polypeptide of claim 4, wherein the peptide or polypeptide is conjugated to a non-natural moiety, a stabilizing or targeting peptide or moiety, a pore forming molecule or a fungicidal molecule.

6. The peptide or polypeptide of claim 5, wherein the non-natural moiety is a fusion protein.

7. The peptide or polypeptide of claim 5, wherein the pore forming molecule is mellatin.

8. The peptide or polypeptide of claim 5, wherein the fungicidal molecule is Fluconazole or Amphotericin B.

9. The peptide or polypeptide of claim 1, wherein said peptide or polypeptide comprises at least one D amino acid or comprises all D amino acids.

10. The peptide or polypeptide of claim 1, further comprising a cysteine in the peptide, wherein the cysteine is positioned at the N-terminus, the C-terminus or in both of these positions.

11. The peptide or polypeptide of claim 1, wherein the peptide or polypeptide is circularized

12. The peptide or polypeptide of claim 1, wherein the peptide or polypeptide is no more than 10 amino acids long, 11 amino acids long, 12 amino acids long, or 13 amino acids long.

13. The peptide or polypeptide of claim 1, wherein the peptide or polypeptide is not circularized and/or is not N-terminally modified.

14. A composition comprising a peptide or polypeptide of any one of claims 1-13 in a pharmaceutically acceptable carrier.

15. The composition of claim 14, wherein composition is frozen or lyophilized.

16. An isolated polynucleotide molecule consisting or consisting essentially of a nucleic acid sequence encoding an amino acid sequence selected from SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, and SEQ ID NO: 12.

17. The polynucleotide molecule of claim 16, wherein the nucleic acid sequence encoding the peptide is operably linked to a promoter, such as in an expression cassette or expression vector.

18. A host cell comprising a polynucleotide molecule of claim 16 or 17.

19. A method of manufacturing a recombinant polypeptide comprising:
(a) expressing a polynucleotide molecule according to claim 16; and
(b) isolating the polypeptide from the cell.

20. A method of treating or preventing a fungal disease in a subject comprising administering an effective amount of a peptide or polypeptide of any one of claims 1-13 or a composition of claim 14 or 15.

21. The method of claim 20, wherein the peptide, polypeptide or composition is administered via a route selected from oral, topical, vaginal, intranasal, intraperitoneal, parenteral, intravenous, intramuscular, subcutaneous, intrathecal, transcutaneous, nasopharyngeal, or via transmucosal absorption, such as by intravenous injection or catheter delivery.

22. The method of claim 20, wherein the fungal disease is caused by an organism in the Candida, Aspergillus, Histoplasma, Cryptococcus, Coccidioides, Paracoccidioides, Blastomyces, Mucor, Rhizopus, Scedosporium, Pneumocystis, Penicillium, Fusarium, Tinea, Malassezia, Trichophyton, Microsporum, or Epidermophyton genera.

23. The method of any one of claims 20-23, wherein the subject is human or a non-human mammal.

24. A composition comprising the peptide or polypeptide of any one of claims 1-13 formulated for application to a surface or a consumable product.

25. The composition of claim 24, wherein the composition is in the form of a paint or coating.

26. The composition of claim 25, wherein the coating is an architectural coating, an industrial coating, or a specification coating.

27. The composition of claim 24, wherein the composition is part of a multicoat system.

28. The composition of claim 24, wherein the composition comprises a binder, such as a thermoplastic binder, a thermosetting binder, or a combination thereof.

29. A method of inhibiting fungal growth on a surface or in a consumable product comprising contacting the composition any one of of claims 24-28 with a surface or consumable product.

30. The method of claim 29, wherein the consumable product is a foodstuff or a liquid.

31. A kit comprising the peptide or polypeptide of any one of claims 1-13 or the composition of any one of claims 14-15 or any one of claims 24-28.


SEQUENCE LISTING (29 sequences)

SEQ ID NO: 1 - protein, 20 aa, synthetic construct
TNVGALWICGLAVIQAAQLS

SEQ ID NO: 2 - protein, 20 aa, synthetic construct
TNAAAAQQGGIICLLVVWLS

SEQ ID NO: 3 - protein, 18 aa, unidentified
TNAIIVAGQLALWAVQCG

SEQ ID NO: 4 - protein, 12 aa, unidentified
VAGQLALWAVQC

SEQ ID NO: 5 - protein, 11 aa, unidentified
AGQLALWAVQC

SEQ ID NO: 6 - protein, 10 aa, unidentified
GQLALWAVQC

SEQ ID NO: 7 - protein, 10 aa, synthetic construct
VVLALWAVQC

SEQ ID NO: 8 - protein, 10 aa, synthetic construct
GQLVVWALQC

SEQ ID NO: 9 - protein, 10 aa, synthetic construct
LVLAFWAVVC

SEQ ID NO: 10 - protein, 10 aa, synthetic construct
VQLALWAVQC

SEQ ID NO: 11 - protein, 10 aa, synthetic construct
VLLALWALVC

SEQ ID NO: 12 - protein, 10 aa, synthetic construct
VQLALWALVC

SEQ ID NO: 13 - protein, 68 aa, Enterococcus faecalis
SDQLEDSEVEAVAKGLEEMYANGVTEDNFKNYVKNNFAQQEISSVEEELNVNISDASTVVQARFNWNA

SEQ ID NO: 14 - protein, 68 aa, Enterococcus faecalis
LGSCVANKIKDEFFAMISISAIVKAAQKKAWKELAVTVLRFAKANGLKTNAIIVAGQLALWAVQCGLS

SEQ ID NO: 15 - protein, 20 aa, Enterococcus faecalis
TNAIIVAGQLALWAVQCGLS

SEQ ID NO: 16 - protein, 10 aa, synthetic construct
AQLALWAVQC

SEQ ID NO: 17 - protein, 10 aa, synthetic construct
GALALWAVQC

SEQ ID NO: 18 - protein, 10 aa, synthetic construct
GQAALWAVQC

SEQ ID NO: 19 - protein, 10 aa, synthetic construct
GQLAAWAVQC

SEQ ID NO: 20 - protein, 10 aa, synthetic construct
GQLALWAAQC

SEQ ID NO: 21 - protein, 10 aa, synthetic construct
GQLALWAVAC

SEQ ID NO: 22 - protein, 11 aa, synthetic construct
AAQLALWAVQC

SEQ ID NO: 23 - protein, 11 aa, synthetic construct
AGALALWAVQC

SEQ ID NO: 24 - protein, 11 aa, synthetic construct
AGQAALWAVQC

SEQ ID NO: 25 - protein, 11 aa, synthetic construct
AGQLAAWAVQC

SEQ ID NO: 26 - protein, 11 aa, synthetic construct
AGQLALWAAQC

SEQ ID NO: 27 - protein, 11 aa, synthetic construct
AGQLALWAVAC

SEQ ID NO: 28 - protein, 12 aa, synthetic construct
VVGQLVLWVVQC

SEQ ID NO: 29 - protein, 12 aa, synthetic construct
VLGQLLLWLVQC